(19) United States
(12) Patent Application Publication
(10) Pub. No.: US 2023/0407267 A1
(43) Pub. Date: Dec. 21, 2023

(54) USES OF PATIENT-DERIVED SCAFFOLDS

(71) Applicant: Iscaff Pharma AB, Göteborg (SE)

(72) Inventors: Göran Landberg, Halmstad (SE); Anders Ståhlberg, Kållered (SE)

(21) Appl. No.: 18/251,326

(22) PCT Filed: Oct. 26, 2021
(86) PCT No.: PCT/SE2021/051076

§ 371 Date: May 1, 2023

(30) Foreign Application Priority Data
Nov. 3, 2020 (SE) 2051274-5

(51) Int. Cl. C12N 5/09

(52) U.S. Cl. CPC C12N 5/0693; C12N 2503/04; C12N 2533/90

(57) ABSTRACT

Cell-free scaffolds derived from tumors in patients are used as in vitro cancer models and also provide information of the tumor, from which it is derived, including its susceptibility to cancer treatment. The cell-free scaffolds are also used as a predictive tool in assessing cancer treatment efficacy and in identifying immunotargets and biomarkers for cancer therapy.

### TECHNICAL FIELD

The present invention generally relates to patient-derived cell-free scaffolds and usages thereof in in vitro models and methods of assessing cancer treatments and analysis of tumors.

### BACKGROUND

Cancer is a major societal challenge that affects an increasing number of people. Breast cancer, for example, affects approximately one out of eight women. Besides the difficulties associated with prolonged treatment periods and related side effects, there is a substantial risk that a cancer, such as breast cancer, will spread and cause metastatic disease. Despite a slightly improved survival for cancer sufferers in general there are major drawbacks with the existing therapies and there is substantial under-treatment due to lack of efficient therapies. These therapy resistant subpopulations of cancer cells are responsible for malignant properties and need to be controlled in order to prevent disease recurrences. Another problem with the existing treatment schedules for cancers is over-treatment due to lack of treatment predictive information guiding clinicians in treatment decision and choices.

WO 2018/083231 discloses a method for determining one or more tumor properties in a subject with a tumor. The method comprises seeding a cell-free scaffold obtained from the tumor with cancer cells and culturing the cancer cells in the scaffold. The method also comprises assaying the cultured cancer cells for the presence of target molecules indicative of the expression of one or more genes in the cells and determining one or more tumor properties based on the results of the assay.

There is still a need for improvement within the field of cancer diagnosis and treatment, in particular regarding knowledge of the immune response modulation by tumor microenvironment.

### SUMMARY

It is a general objective to use patient-derived cell-free scaffolds in in vitro models and methods of assessing cancer treatments and analysis of tumors.

The present invention is defined in the independent claims. Further embodiments of the invention are defined in the dependent claims.

An aspect of the invention relates to a method of providing an in vitro cancer model. The method comprises selecting a cell-free scaffold obtained from a tumor in a subject. The tumor has at least one defined tumor property and the cell-free scaffold is selected based on the at least one defined tumor property. The method also comprises selecting cancer cells based on the selected cell-free scaffold. The selected cancer cells are not obtained from the tumor from which the cell-free scaffold is obtained. The method further comprises culturing the selected cancer cells in the selected cell-free scaffold.

Another aspect of the invention relates to a method for identifying an immunotarget for cancer therapy for a subject. The method comprises culturing cancer cells in a cell-free scaffold obtained from a tumor in the subject. The method also comprises analyzing expressions of immunomarkers in the cancer cells and selecting at least one immunotarget for cancer therapy for the subject based on the expressions of immunomarkers in the cancer cells.

A further aspect of the invention relates to a method of determining a tumor-specific immunomarker. The method comprises culturing cancer cells in a cell-free scaffold obtained from a tumor in the subject and analyzing expressions of immunomarkers in the cancer cells. The method also comprises comparing the expressions of the immunomarkers with control levels of expressions of immunomarkers and determining the tumor-specific immunomarker for the tumor in the subject based on the comparison.

Yet another aspect of the invention relates to a method of determining at least one tumor property of a tumor in a subject. The method comprises culturing cancer cells, preferably cancer cells, in a cell-free scaffold obtained from a tumor in the subject. The method also comprises analyzing the expression of at least one immunomarker selected from the group consisting of programmed death ligand 1 (PDL1), colony stimulating factor 1 (CSF1) and chemokine C—C motif ligand 2 (CCL2) in the cancer cells. The method further comprises determining the at least one tumor property based on the expression of the at least one immunomarker.

The invention also relates to a method of determining susceptibility of a tumor to immunotherapy. The method comprises co-culturing cancer cells and leukocytes in a cell-free scaffold obtained from a tumor in a subject. The method also comprises determining the viability of the cancer cells and determining the susceptibility of the tumor in the subject to immunotherapy based on the determined viability of the cancer cells.

Another aspect of the invention relates to a method of determining at least one tumor property of a tumor in a subject. The method comprises co-culturing cancer cells and leukocytes in a cell-free scaffold obtained from a tumor in a subject. The method also comprises analyzing the expression of at least one tumor property marker in the viable cancer cells and determining at least one tumor property based on the expression of the at least one tumor property marker in the viable cancer cells co-cultured with the leukocytes in the cell-free scaffold.

A further aspect of the invention relates to a method of determining susceptibility of a tumor to immunotherapy. The method comprises co-culturing cancer cells and leukocytes in a cell-free scaffold obtained from a tumor in a subject. The method also comprises analyzing the expression of at least one immunomarker in the cancer cells from the co-culture of cancer cells and leukocytes in the cell-free scaffold and determining the susceptibility of the tumor in the subject to immunotherapy based on the expression of the at least one immunomarker.

Yet another aspect of the invention relates to a method of determining a biomarker for susceptibility of a tumor to immunotherapy. The method comprises culturing cancer cells in a cell-free scaffold obtained from a tumor in a subject and analyzing the expression of at least one target molecule in viable cancer cells from the culture of cancer cells in the cell-free scaffold. The method also comprises co-culturing cancer cells and leukocytes in a cell-free scaffold obtained from a tumor in the subject and analyzing the expression of the at least one target molecule in viable cancer cells from the co-culture of cancer cells and leukocytes in the cell-free scaffold. The method further comprises comparing the expression of the at least one target molecule in the viable cancer cells from the culture with the expression of the of the at least one target molecule in the viable cancer cells from the co-culture and determining, among the at least one target molecule, a biomarker for susceptibility of the tumor to immunotherapy based on the comparison.

An additional aspect of the invention relates to a method of determining efficacy of a cancer treatment for a subject having a tumor. The method comprises a) co-culturing cancer cells and leukocytes in a cell-free scaffold obtained from a tumor in the subject and b) analyzing the expression of at least one target molecule in the cancer cells. The method also comprises c) exposing the cancer cells to a cancer treatment when co-cultured with leukocytes in the cell-free scaffold and d) analyzing the expression of the at least one target molecule in the cancer cells after exposure to the cancer treatment. The method further comprises e) comparing the expressions of the at least one target molecule analyzed in b) and d) and f) determining efficacy of the cancer treatment based on the comparison.

A related aspect of the invention defines a method of determining efficacy of a cancer treatment for a subject having a tumor. The method comprises a) co-culturing cancer cells and leukocytes in a cell-free scaffold obtained from a tumor in the subject before the cancer treatment has been applied to the subject and b) analyzing the expression of at least one target molecule in the cancer cells. The method also comprises c) co-culturing cancer cells and leukocytes in a cell-free scaffold obtained from a tumor in the subject after the cancer treatment has been applied to the subject and d) analyzing the expression of the at least one target molecule in the cancer cells. The method further comprises e) comparing the expressions of the at least one target molecule analyzed in b) and d) and f) determining efficacy of the cancer treatment based on the comparison.

The present invention further relates to a tumor scaffold comprising a cell-free scaffold obtained from a tumor in a subject, cancer cells cultured in the cell-free scaffold and leukocytes co-cultured with the cancer cells in the cell-free scaffold.

Cell-free scaffolds derived from tumors in patients are useful not only as in vitro cancer models but also provides patient-specific, or more correctly tumor-specific, information of the tumor, from which it is derived, including its susceptibility to cancer treatment. The cell-free scaffolds can be used as predictive tool in assessing cancer treatment efficacy and in identifying immunotargets and biomarkers for cancer therapy. Hence, the cell-free scaffolds can be used as an in vitro tool mimicking in vivo tumor properties and the local tumor microenvironment. Treatment predictive markers for novel cancer therapies are needed in order to increase the precision of the treatment and identify the patients that benefits of the therapies. The cancer microenvironment as represented by the cell-free scaffolds and its immunomodulating effects has potential to indeed provide treatment predictive information for immune targeting cancer therapies.

### BRIEF DESCRIPTION OF THE DRAWINGS

The embodiments, together with further objects and advantages thereof, may best be understood by making reference to the following description taken together with the accompanying drawings, in which:

FIG. 1. Cancer cells growing in patient-derived cell-free scaffolds (PDSs) show patient-dependent expression of certain immune markers. MCF7 or MDA-MB-231 cell lines were grown for 21 days in 101 and 85 PDSs, respectively, originating from biobanked tumor samples. RNA levels of 6 immune markers (CCL2, CD47, CSF1, PDL1, MR1 and PDL2) were analyzed by qPCR, relativized against 2D cultures and represented in log₂. (A) and (B) Scatter plots where each dot represents the individual expression of each gene for each PDS. (C) and (D) Heat maps showing the expression of each gene (rows) for each PDS (columns).

FIG. 2. Representations of the relationship between expression of PDL1, CSF1 and CCL2 immunomarkers in MCF7 cells growing in PDSs to relevant clinical variables. (A, B, D, F) Box plots min to max representing the median and the spread of the PDS-induced expression values for PDL1, CSF1 and CCL2. Mann-Whitney U statistical test was applied and p-value<0.05 were considered significant (*p-value<0.05, **p-value<0.01). (C, E, G) Kaplan-Meier plots illustrating PDS-induced PDL1 and CSF1 expression in relation to disease free survival (DFS), or CCL2 expression in relation to recurrences, respectively. Long-rank statistical test was applied and p-value<0.05 were considered significant (*p-value<0.05, **p-value<0.01).

FIGS. 3A-3D. Expression of 183 genes related with immune response and modulation expressed by MCF7 cells growing in PDSs. MCF7 cells were grown for 21 days in a cohort of 48 breast cancer PDSs. Then global gene expression was analyzed by next generation sequencing (NGS) and 183 out of 399 immune markers were detected. Graphs show scatter plots representing the individual expression of each gene in each PDS. The 399 gene list was a compendium generated from data available in OLINK, GSEA and bibliography.

FIGS. 4A and 4B. The expression of CCL5 and ETS1 immune markers in MCF7 growing in PDSs are associated to relevant clinical variables. PDS-dependent induction of low and high levels of CCL5 and ETS1 respectively was associated to ER-, PR- and metastasis in lymph nodes (LN). Box plots min to max representing the median and the spread of the PDS-induced expression values for CCL5 and ETS1. Mann-Whitney U statistical test was applied and p-value<0.05 were considered significant.

FIGS. 5A-5C. PDS expression profiles for immune marker genes. MCF7 cells were grown 21 days in a cohort of 48 breast cancer PDSs. The global gene expression was analyzed by NGS and 183 of 399 immune markers were detected. Principal Components Analysis (PCA) was then applied to the 183 genes. (A) PCA scores show that the PDSs are distributed in 2 clusters (identification number is showed next to the each PDS). (B) PCA gene loading illustrating the contribution to the PCA scores in (A). Black dots in (B) indicate the 30 immune markers with the highest influence on the PDS cluster distribution. These 30 genes were then identified using self-organizing map (SOM) and dynamic PCA tools. (C) Heatmap showing the expression of the 30 immune markers from (B) (columns) along the different PDSs (rows) using average linkage as clustering method and Euclidean distances as the distance measure. The 14 PDSs (columns) grouped on the left coincide with the left cluster in (A).

FIG. 6. The patient-dependent expression of the immune markers in HT29 cancer cells growing in colon cancer PDS samples are associated to relevant clinical variables. The HT29 cell line was grown for 21 days in 80 colon cancer PDSs, originated from biobanked tumors. mRNA levels of 4 immune markers (CCL2, CD47, CSF1 and PDL1) were analyzed by qPCR, relativized against 2D cultures, and represented in log₂. (A) Scatter plots representing the individual expression of each gene in each PDS (CCL2 expression was not detected). (B-D) PDS-dependent induction of high levels of CSF1 mRNA were associated to high stage (B), relapses (C) and with short periods free of disease (DFS, Disease Free Survival) (D). (B, C) Box plots min to max representing the median and the spread of the PDS-induced expression values for CSF1. Mann-Whitney U statistical test was applied and p-value<0.05 were considered significant (*p-value<0.05, **p-value<0.01). (D) Kaplan-Meier plot illustrating PDS-induced CSF1 expression in relation to DFS. Long-rank statistical test was applied and p-value<0.05 were considered significant (*p-value<0.05, **p-value<0.01).

FIG. 7. PDSs contribute to the activation of the T cells increasing the CD69 expression in pre-activated T cells, but only the presence of cancer cells influenced the fraction of PD1 expressing T cells. After co-culture with MCF7/Luc cells, T cells were harvested and the surface markers CD3, CD4, CD8, CD69, CD25 and CD127 were analyzed by FACS. Non-activated (TNA) and activated T cells (TA+) without being in contact with MCF7/Luc were used as controls. (A, B) % of T cells presenting CD69 marker. (C, D) % of T cells presenting PD1 receptor. (E) % T reg cells (CD4+ T cells presenting CD25high CD127low). Average and standard deviation of 3-12 biological replicates are shown.

FIG. 8. Activated T cells are able to kill MCF7/Luc cells promoting enrichment of PDL1-high expressing MCF7/Luc cells. (A) Viability of MCF7/Luc cells and (B) relative PDL1 mRNA levels (log₂) in MCF7/Luc cells remained after co-cultures with non-activated T cells (TNA) or activated T cells (TA+), in 2D or in PDS cultures. Cultures of MCF7/Luc without T cells in both 2D and PDS were used as viability (value set to 100%) and expression references (value set to 0). Average and standard deviation of 3-12 biological replicates are shown.

FIGS. 9A and 9B. Co-cultures with active T cells enrich for MCF7/Luc cells with low-proliferative and low-differentiated phenotype and high cancer stem features. The figure shows differences in expression (represented in log₂) of a gene panel representative of relevant breast cancer related processes (proliferation, differentiation, pluripotency, EMT, BCSC and apoptosis) in MCF7/Luc cells after 72 hours of co-cultures, comparing cultures with non-activated T cells (TNA) or activated T cells (TA+), and 2D and PDS cultures. Culture of MCF7/Luc cells without T cells in 2D or PDS was included as a control and different passages of MCF7/Luc were used to normalize the data. Average and standard deviation of 2-4 biological replicates are shown.

FIG. 10. The cancer cell killing capacity of non-activated (TNA) and activated T cells (TA-F) is PDS-dependent. (A) Viability of MCF7/Luc cells after 72 hours of co-cultures with non-activated T cells (TNA) in 12 different PDSs. (B) Viability of MCF7/Luc cells after co-culture with activated T cells (TA-F) following different activation protocols and co-culture incubations in 12 different PDSs. Culture of MCF7/Luc cells without T cells in each individual PDS was used as a control and as viability reference (viability in control set to 100%) for A and B.

FIG. 11. Viability of MCF7/Luc cells (A) and IFNγ released to the media by the T cells (B) after 72 hours of co-cultures with non-activated T cells (TNA) or strongly activated T cells (TA-F) (48 hours with CD3/CD28 Dynabeads) in 4 different PDSs. Single culture of MCF7/Luc in each individual PDS was used as a control and as viability reference 100%.

FIG. 12. Immunofluorescence staining of the breast cancer PDS cultures of MCF7 cancer cells with non-active or active T-lymphocytes. Breast cancer PDSs were re-cellularized for 21 days with MCF7 cells, afterwards T-lymphocytes were added and incubated together for 48 hours. T-lymphocytes were activated by exposition to CD3/CD28 Dynabeads during the previous 20 hours before addition to the cultures. Antibodies against Pan-CK were used to detect cancer cells, against CD3 to detect T-lymphocytes (T cells) and against PD-L1.

FIG. 13. The cancer cell killing capacity correlates with the PDS induction of PDL1 expression in MCF7/Luc cells. (A) Relative PDL1 mRNA levels (log₂) in MCF7/Luc cells after 21 days in different PDSs prior to T cell addition (basal PDL1 expression). 2D MCF7/Luc cultures were used to normalize the data. (B, C) Scatter plots of basal PDL1 expression in MCF7/Luc cells growing in 12 different PDSs in correlation to their viability after co-culture with non-activated T cells (TNA) (B) or activated T cells (TA+) (C). The trend line is plotted and the Pearson's coefficient correlation and p-value indicated.

FIG. 14 is a flow chart illustrating a method of providing an in vitro cancer model according to an embodiment.

FIG. 15 is a flow chart illustrating a method for identifying an immunotarget for cancer therapy according to an embodiment.

FIG. 16 is a flow chart illustrating a method of determining a tumor-specific immunomarker according to an embodiment.

FIG. 17 is a flow chart illustrating a method of determining at least one tumor property of a tumor according to an embodiment.

FIG. 18 is a flow chart illustrating a method of determining susceptibility of a tumor to immunotherapy according to an embodiment.

FIG. 19 is a flow chart illustrating a method of determining at least one tumor property of a tumor according to another embodiment.

FIG. 20 is a flow chart illustrating a method of determining susceptibility of a tumor to immunotherapy according to another embodiment.

FIG. 21 is a flow chart illustrating a method of determining a biomarker for susceptibility of a tumor to immunotherapy according to an embodiment.

FIG. 22 is a flow chart illustrating a method of determining efficacy of a cancer treatment for a subject having a tumor according to an embodiment.

FIG. 23 is a flow chart illustrating a method of determining efficacy of a cancer treatment for a subject having a tumor according to another embodiment.

FIG. 24. Illustrative schema of PDS co-cultures of cancer cells and macrophages. PDS is re-cellularized with a cancer cell line, the PDS microenvironment trigger the expression and production cytokines, such as CSF1 and CCL2, that are involved in the recruitment, survival and polarization of the macrophages. Monocyte THP-1 cells are added to the PDS culture and they will polarize to M1- or M2-like macrophages. The proportion of M1/M2-like macrophages will be characteristic of each PDS, where factors coming from the cancer cells, including, but not limited to secreted cytokines and receptors exposed in the cell surface, and from the PDS per se, including, but not limited to structure, and attached molecules, will affect the macrophage polarization. The PDS system will be used as testing platform for therapies targeting macrophages polarization. Also, it will give predictive information for a specific patient by including therapy screens, and contribute to the design of new personalized treatment strategies by targeting the TAM-mediated immune suppression.

FIG. 25. PD1 blocking antibody increases the killing capacity of the T-lymphocytes in PDS-dependent manner. T-lymphocytes were activated 20 hours by exposition to CD3/CD28 Dynabeads. Afterwards, some of the T-lymphocytes were treated with PD1 blocking antibody Pembrolizumab (Pembr or PD1Ab) for 30 min (100 μg/mL in 1×10⁶ T-lymphocytes/mL suspension) before they were added to PDS cultures re-cellularized with MCF7 cells for 21 days. Then, the co-cultures were incubated for 48 hours. Controls including only MCF7 cells or T-lymphocytes, and 2D cultures were included. (A) Surface expression of PD1 by T-lymphocytes analyzed by FACS. (B) Graph showing the % of viable MCF7 cells after 48 hours of co-culture with T-lymphocytes in 5 different PDSs. Data are relative to their own control with only MCF7 cells and show average±SD of 3 technical replicates.

### DETAILED DESCRIPTION

The present invention generally relates to patient-derived cell-free scaffolds and the usage thereof in in vitro models and methods of assessing cancer treatments and analysis of tumors.

Patient-derived cell-free scaffolds (PDSs) derived from tumors in patients are useful not only as in vitro cancer models but also provides patient-specific, or more correctly tumor-specific, information of the tumor, from which it is derived, including its susceptibility to cancer treatment. The PDSs can be used as a predictive tool in assessing cancer treatment efficacy and in identifying immunotargets and biomarkers for cancer therapy. Hence, the PDSs can be used as an in vitro tool mimicking in vivo tumor properties and the local tumor microenvironment. The PDS may thereby be used to assess, for instance, the efficacy of a cancer treatment in vitro but where the determined efficacy predicts the outcome of the cancer treatment if applied to the given patient, from which the PDS is derived. Hence, PDSs find uses in individualized or patient-specific cancer treatment.

The complexity of the in vivo tumor microenvironment and its effects on tumor properties, such as tumor growth and susceptibility to cancer treatment, including immunotherapy, differs to most model systems used in cancer research today. The in vitro models used are typically represented by two dimensional (2D) cell cultures of cancer cell lines growing on plastics under high oxygen supply and immense growth factor activation. Also 3D cell cultures can be used as in vitro models to more accurately mimic the natural growth conditions of cancer cells in a tumor as compared to 2D cell cultures. Examples of such in vitro 3D cell cultures of cancer cells include spheroids and organoids. Spheroids are spherical cellular units generally cultured as free-floating aggregates. Spheroids are, however, of low complexity and do not represent relevant tumor organization. Organoids resemble the original tumor tissue both histologically and genetically, but they do not reconstitute the complex tumor microenvironment. None of these in vitro 3D models are, thus, able to recapitulate the tumor microenvironment of the original tumor. The in vivo animal models, using mainly immunocompromised mice, at least in part create more in vivo like cancer growth conditions by the use of implanted human tumors in the form of xenografts. Compared with the in vitro models, such in vivo model systems can be used for drug testing and studies of cancer growth in a more complex environment, but they have several limitations associated with immunocompromised mice as well as non-human stromal reactions. For instance, breast cancer cell growth in xenografts does not mimic in vivo growth in patients as the cells tend to be less infiltrative and also to have large central necrotic areas due to rapid cell division in relation to angiogenic support. This creates an artificial cancer growth system that might be superior to less complex cell cultures but is still not close enough to real in vivo conditions.

In contrast to these animal models, the PDSs of the present invention mimic in vivo tumor conditions. For instance and as is shown herein, PDSs induce patient or tumor specific expression of immune markers when cancer cells are cultured on the PDSs, demonstrating the difference in tumor properties and microenvironment between different patients although suffering from a same type of cancer, such as breast cancer. Furthermore, such PDS-specific induction of gene expression is correlated with clinical variables of the original tumor including tumor grade and tumor recurrence. The PDSs also affect the efficacy of immunotherapy as assessed when co-culturing cancer cells and leukocytes in the PDSs.

Hence, although devoid of all original cancer cells, the PDS maintains or mimics properties of the tumor microenvironment and it can thereby be used to provide valuable information, including patient-specific or tumor-specific information, relating to the tumor, its immune capacity and susceptibility to various forms of cancer treatment. Such information can be deduced from the (genetic and/or protein) composition of the PDS itself, and/or from changes in gene expression induced by the PDS in cancer cells, or in other cells, when cultured in the PDS. This should be compared to synthetically derived 3D scaffolds providing an artificial environment that is different from the microenvironment of a tumor. Such synthetically derived 3D scaffolds will thereby not mimic the patient-specific or tumor-specific microenvironment and will thereby not enable deriving patient-specific or tumor-specific information.

PDSs derived from different patients may have generic properties that are common for the PDSs and thereby the tumors and patients, from which they are derived. However, PDSs also have unique properties in terms of being patient specific, or more correctly tumor specific, i.e., reflecting the properties of the particular tumor microenvironment of the tumor.

A PDS, as referred to herein, is a cell-free scaffold obtained from a tumor in a subject or patient. The extracellular matrix of a tumor generally comprises a collection of extracellular molecules, including proteins, secreted by cells that provides structural and/or biochemical support to the surrounding cells. The network of extracellular molecules constitutes a 3-dimensional (3D) scaffold for cells in the tumor. Typically, the scaffold provides a microenvironment for the tumor cells, with which the cells can interact. A tumor scaffold may comprise, for example collagen and various tumor promoting factors as growth factor as well as inhibitors affecting tumor cell behaviors. A cell-free scaffold generally refers to decellularized tumor tissue. A cell-free scaffold comprises decellularized extracellular material obtained from the tumor, in which the original 3D structure is substantially preserved. The bioactivity of the cell-free scaffold is substantially preserved. A cell-free scaffold allows effective attachment, migration, proliferation and 3D organization of cells cultures therein. Generally the decellularized scaffold is substantially free of cells, in particular tumor cells. Hence, the PDS is substantially free from cancer cells. In other words, a PDS obtained from a tumor of a patient preferably does not comprise any cancer cells otherwise present in the tumor, from which the PDS is obtained. This may be assessed by any suitable means. Merely by way of example, sectioning and microscopic visualization may be used to determine the presence of absence of nuclei, which are indicative of cells, or DNA analysis may be used. Substantially free of cells means that cells are not detectable in the assessments.

A sample comprising a scaffold from a tumor may be prepared using methods known in the art from, for example, a biopsy. A cell-free scaffold may be obtained from a tumor using suitable decellularizing methods to remove cells, while preserving the basic tumor scaffold composition. A suitable method is disclosed in Example 1. For example, decellularizing methods often employ a prolonged mild detergent treatment.

Merely by way of example, a decellularizing method may comprise subjecting a suitable tumor sample, such as a section taken from a tumor sample, to one or more, for instance two, three, four or more, detergent or lysis washes, often referred to as decellularization cycles. Any suitable detergent may be used in the detergent or lysis buffer including, but not limited to SDS (sodium dodecyl sulfate), Triton X-100 (2-[4-(2,4,4-trimethylpentan-2-yl)phenoxy]ethanol), NP40 (nonyl phenoxypolyethoxylethanol), and/or TWEEN 20® (polyoxyethylene (20) sorbitan monolaurate). Optionally, after each cycle, a small tissue extract may be screened for the presence or absence of cells, for example, by screening for nuclei. Typically, the decellularization cycles are continued until cells are not detectable. Decellularization cycles may be followed by one or more washes to remove cell debris using, for instance, distilled water, a buffer solution or the detergent or lysis buffer but excluding the detergent. A cell-free scaffold may be sterilized using a suitable sterilizing agent, including, but not-limited to, peracetic acid, antibiotics, etc.

A tumor as referred to herein may be any suitable tumor obtained from a subject. The tumor may be benign or malignant, preferably a malignant tumor. A tumor may be of any suitable tissue. In an embodiment, the tumor is a solid tumor. Illustrative, but non-limiting, examples of solid tumors include sarcomas, carcinomas and lymphomas. In a particular embodiment, the solid tumor is obtained from a subject suffering from a cancer disease selected from the group consisting of breast cancer, lung cancer, prostate cancer, colon cancer, skin cancer, liver cancer, ovarian cancer, urinary bladder cancer, esophageal cancer, and pancreatic cancer. Currently preferred solid tumors are a breast cancer tumor and a colon cancer tumor, in particular a breast cancer tumor. Experimental data as presented herein indicates that the PDS of the embodiments can be used as a tumor model for solid tumors of different cancer types.

The tumor sample used to derive the PDS is obtained from a subject. A subject as used herein includes a human subject, such as a male subject or a female subject. The embodiments are, however, not limited to human subjects but can also use PDSs derived from non-human animals, in particular non-human mammals. Illustrative, but non-limiting, examples of such non-human mammals include dogs, cats, mice, rats, goats, sheep, cattle, horses, guinea pigs, rabbits, pigs and primates.

As briefly mentioned in the foregoing, PDSs induce subject-dependent or subject-specific expression of immune markers when cancer cells, not originating from the tumor itself, are cultured on the PDSs, demonstrating the differences in tumor properties and microenvironment between PDSs and tumors derived from different subjects. Hence, a particular PDS can be selected to be used in an in vitro cancer model to mimic desired tumor properties of the tumor, from which it is obtained. Furthermore, experimental data as presented herein shows that different types of cancer cells respond differently when cultured in PDSs. Hence, cancer cells are more or less sensitive to the influence of the microenvironment of a PDS and thereby more or less suitable for usage in such an in vitro cancer model.

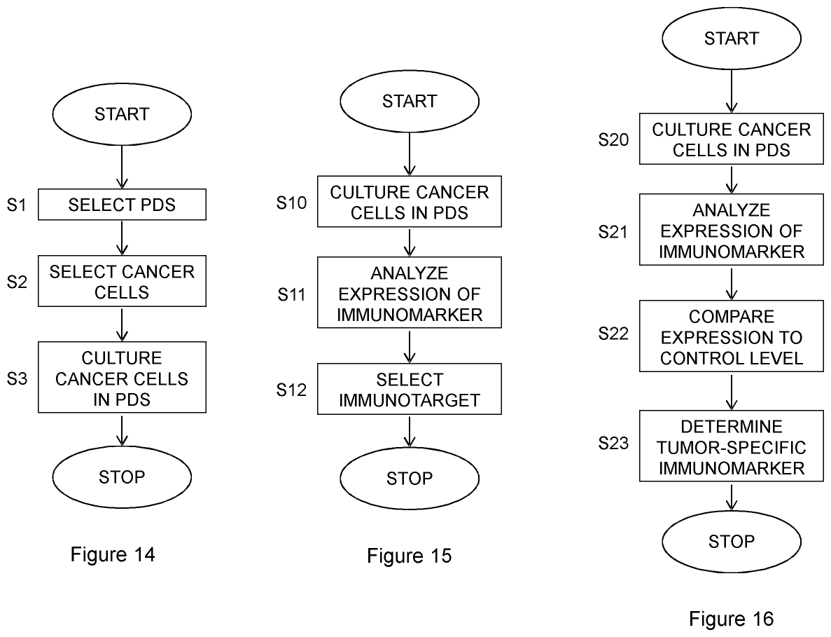

An aspect of the invention relates to a method of providing an in vitro cancer model, see FIG. 14. The method comprises selecting, in step S1, a cell-free scaffold obtained from a tumor in a subject. The tumor has at least one defined tumor property and the cell-free scaffold is selected based on the at least one defined tumor property. The method also comprises selecting, in step S2, cancer cells based on the selected cell-free scaffold. The selected cancer cells are not obtained from the tumor, from which the cell-free scaffold is obtained. The method further comprises culturing, in step S3, the selected cancer cells in the selected cell-free scaffold.

Hence, the in vitro cancer model is provided by selecting, in step S1, a PDS obtained from a tumor having at least one desired tumor property, also referred to as clinical variable of the tumor herein.

Generally, tumors have properties characterizing the tumor including its malignancy, which is typically defined by tumor grades or tumor malignancy grades; cancer type, which defines the cancer disease the tumor is causing, such as breast cancer type; tumor location, i.e., the tissue or organ from which the tumor originates; differentiation capability; proliferation capability; invasiveness, i.e., infiltration capacity; metastasizing capability; epithelial-mesenchymal transition (EMT) capability; and cancer stem cell (CSC) capability.

For instance, a tumor may be assigned a particular grade, with higher grade indicating a more aggressive tumor. Tumour (malignancy) grade is usually assigned according to the appearance of the tumor cells, for example under a microscope. Grading systems for tumors are known to the skilled person. Higher grade tumors are sometimes referred to as progressive tumors. A progressive tumor is generally more aggressive than a non-progressive tumor. Typically, a progressive tumor has one or more of increased invasiveness, higher malignancy grade or malignancy potential, increased risk of recurrence, increased resistance to treatment, and/or increased tumor proliferation, compared to a non-progressive tumor.

Generally, tumor property as used herein refers to any clinically relevant characteristics of a tumor. Tumor properties may be those associated with, or indicative of, a progressive tumor. Such tumor properties may be those which are significant in determining tumor progression, for example, properties which are useful for distinguishing progressive tumors from non-progressive tumors. Suitable tumor properties may include, for example, invasiveness, migration, malignancy grade or malignancy potential, risk of recurrence, resistance to treatment, and/or tumor proliferation.

Hence, in an embodiment, the tumor has at least one defined tumor property selected from the group consisting of tumor malignancy grade, cancer type, tumor location, differentiation capability, proliferation capability, infiltration capability, metastasizing capability, EMT capability and CSC capability.

In an embodiment, the method also comprises determining the at least one tumor property of the tumor, from which the cell-free scaffold is obtained. The determination of the at least one tumor property of the tumor can be performed according methods and techniques well-known in the art. For instance and as mentioned in the foregoing, tumor malignancy grade can be determined based on the appearance of the tumor cells, for example under a microscope. Such visual inspection of tumor cells may also provide information of the differentiation capability, proliferation capability, infiltration capability, metastasizing capability, EMT capability and/or CSC capability of the tumor. Cancer type and tumor location may be determined based on the location of the tumor in the patient body.

In a particular embodiment, determining the at least one tumor property comprises determining at least one tumor property selected from the group consisting of tumor malignancy grade, cancer type, tumor location, differentiation capability, proliferation capability, infiltration capability, metastasizing capability, EMT capability and CSC capability of the tumor, from which the cell-free scaffold is obtained.

In the method, one or more desired tumor properties as exemplified above are selected in step S1 and a tumor having the one or more desired tumor properties is then identified, such as from a subject or a tumor sample, such as biopsy, previously taken from a subject. A PDS is then derived from the selected tumor. Cancer cells are then selected in step S2 based on the selected PDS. The cancer cells selected in step S2 based on the selected PDS and then cultured in the selected PDS in step S3 are not from the tumor, from which the PDS is obtained. As mentioned in the foregoing, the PDS is substantially free from cells, including cancer cells. Step S2 thereby involves selecting cancer cells to be added to and cultured in the PDS. The cancer cells selected in step S2 based on the selected PDS are, in an embodiment, not obtained from the patient nor derived from cancer cells obtained from the patient body. Hence, in an embodiment, the cancer cells selected in step S2 are obtained from a source other than the tumor, and preferably from a source other than the patient.

In an embodiment, step S2 comprises selecting cancer cells based on the at least one defined tumor property of the tumor, from which the selected PDS is obtained. Hence, in such an embodiment, the cancer cells to be cultured in the PDS in step S3 are selected on the at least one defined tumor property of the tumor. For instance, first cancer cells could be selected in step S2 for PDSs obtained from high tumor malignancy grade tumors, whereas second, different cancer cells could be selected in step S2 for PDSs obtained from low tumor malignancy grade tumors. As another example, first cancer cells could be selected in step S2 for PDS obtained from breast cancer tumors, whereas second, different cancer cells are selected in step S2 for PDSs obtained from other types of tumors.

In an embodiment, a cancer cell line is selected in step S2 based on the PDS selected in step S1 and cancer cells of the selected cancer cell line are cultured in the PDS. A cancer cell line as used herein is a population of cancer cells from a tumor that, for instance due to at least one mutation, have evaded the normal cellular senescence and instead can keep undergoing division. Cancer cells of the cancer cell line can therefore be grown for prolonged periods in vitro.

Alternatively, a type of cancer cells is selected in step S2 based on the PDS selected in step S1. The type of cancer cells refers to the particular cancer disease caused by the cancer cells, such as breast cancer cells, prostate cancer cells, etc. It is also possible to select a cancer cell line of a particular type of cancer cells based on the selected PDS, such as selection between breast cancer cells of the MCF7 (luminal A subtype) cell line or of the MDA-MB-231 (Triple Negative B subtype) cell line.

It is also possible to select more than one cancer cell line in step S2 based on the PDS selected in step S1. Hence, step S2 also encompass selecting a combination of two or more cancer cell lines.

In a particular embodiment, primary cancer cells or a primary cancer cell population are or is selected in step S2 based on the PDS selected in step S1. Primary cancer cells or primary cancer cell population as used herein refer to cancer cells or a cancer cell population from a primary tumor as compared to cancer cells or a cancer cell population from metastases.

In an embodiment, the method comprises selecting, in step S2, a cancer cell line or a primary cancer cell population based on the PDS selected in step S1. In such an embodiment, the selected cancer cell line or the primary cancer cell population is cultured in the selected PDS in step S3.

In a particular embodiment, the method comprises selecting, in step S2, cancer cells that are sensitive to the influence of the microenvironment of the PDS selected in step S1. For instance, the MCF7 cell line is more sensitive to the different microenvironments provided in the PDSs as compared to the MDAMB231 cell line. Furthermore, the matching or concordance between a selected PDS and selected cancer cells improves the link or association between, for instance, immunomarkers, also referred to as immune markers herein, expressed by the cancer cells when cultured in the PDS and tumor properties or clinical variables associated with the tumor from which the PDS originates.

Hence, in an embodiment, the method comprises selecting, in step S2, cancer cells having an inducible expression of at least one immunomarker. In such an embodiment, the expression of the at least one immunomarker is altered by the microenvironment of the PDS selected in step S1. Thus, the selected cancer cells express at least one immunomarker when cultured on the selected PDS. Furthermore, the expression of the at least one immunomarker is dependent on, i.e., altered, by the particular microenvironment of the selected PDS. For instance, experimental data as presented herein show that individual PDS significantly affected the expression of various immunomarkers, including programmed death ligand 1 (PDL1), colony stimulating factor 1 (CSF1) and chemokine C—C motif ligand 2 (CCL2), in cancer cells cultured in the different PDSs.

Immunomarker, also referred to as immunotarget herein, includes any biomarker representative of immunoregulatory and/or inflammatory processes in subjects. An immunomarker can be analyzed through the expression of the gene encoding the particular protein involved in the immunoregulatory and/or inflammatory process and/or using immunoassays to detect and optionally quantify the particular protein.

In an embodiment, the method comprises an additional step of adding the selected cancer cells to the selected PDS prior to culturing the cancer cells in the selected PDS in step S3. For instance, cancer cells in culture medium could be added to the selected PDS.

The culturing of the selected cancer cells in the selected PDS in step S3 is typically conducted in accordance with standard in vitro culturing conditions and in a culture medium selected at least partly based on the selected cancer cells. For instance, the culturing conditions may include a culturing temperature of about 37° C. in a 5% CO₂ humidified atmosphere. Illustrative, but non-limiting, examples of culture media that could be used include Plasmax™, Human Plasma-Like Medium (HPLM), Minimal Essential Medium (MEM), Iscove's Modified Dulbecco's Medium (IMDM), Dulbecco's modified Eagles Medium (DMEM), Ham's F-12 Nutrient Mix (F-12), Roswell Park Memorial Institute 1640 Medium (RPMI 1640), and mixtures thereof. The culture medium may optionally be supplemented with various components including, for instance, a serum component, such as fetal bovine serum (FBS), antibiotics, amino acids, vitamins, etc.

In an embodiment, the method comprises analyzing the expression of at least one immunomarker in the cancer cells cultured in the selected PDS.

The microenvironment of the PDS affects, i.e., modulates, the expression of immunomarkers by the cancer cells when cultured in the PDS. Furthermore, this modulation of the expression of immunomarkers provides valuable information of the PDS and thereby of the tumor from which the PDS originates. For instance, experimental data as presented herein shows that there is a correlation between expression of various immunomarkers by cancer cells and tumor properties relevant for the tumor and the cancer disease. This means that the in vitro model can be used as a tool to determine such tumor properties by analyzing the expression of at least one immunomarker in the cancer cells when cultured in the PDS.

The analysis of the expression of the at least one immunomarker can be performed according to various embodiments. Illustrative examples include analysis at the RNA level, such as using a gene expression array that measures the levels of gene products, i.e., mRNA transcripts, from selected genes encoding immunomarkers. Such gene expression arrays are also denoted DNA (micro)array in the art. Alternatively, gene expression can be analyzed using quantitative polymerase chain reaction (qPCR) as disclosed in Example 2 or by gene sequencing. A further alternative is to use an immunoassay, i.e., detecting and preferably quantifying, immunomarkers using antibodies that specifically bind to the respective immunomarkers. Such immunoassays include ELISA, immunostaining, dot blot methods, Western blot and other immunohistochemistry (IHC) and immunocytochemistry (ICC) methods. Further techniques include mass spectrometry (MS), fluorescence-activated cell sorting (FACS), proximity extension assay (PEA), proximity ligation assay (PLA).

In a particular embodiment, the method comprises culturing the selected cancer cells in a 2D culture, such as in the form of a monolayer in a culture flask or Petri dish. The method also comprises analyzing the expression of the at least one immunomarker in the cancer cells cultured in the 2D culture and comparing this expression with the corresponding expression of the least one immunomarker in the cancer cells cultured in the selected PDS. Hence, in this particular embodiment, the difference in expression of the at least one immunomarker between 2D culturing and culturing in the PDS is determined. Such a difference can provide value information of specific properties of the particular PDS and thereby also of tumor properties relevant for the tumor and the cancer disease.

Instead of, or as a complement to, using a 2D culture as reference, the cancer cells could be cultured in multiple, i.e., at least two, but typically a plurality of different PDSs obtained from tumors in multiple subjects. In such a case, the expression of the at least one immunomarker is analyzed in these cancer cells cultured in the different PDSs and used to determine a respective control or reference level of expression of the at least one immunomarker, such as in the form of an average or median expression level in the cancer cells in the different PDSs. The expression of the least one immunomarker in the cancer cells cultured in the selected PDS can then be compared to this control or reference level.

In an embodiment, the method also comprises determining at least one cellular property or characteristic of the cancer cells cultured in the selected PDS.

Cellular property or characteristic as used herein relates to a property or characteristic of the cancer cells cultured in the PDS. Illustrative examples of such cellular property or characteristic include cell morphology, cell size, cellular motility, cellular invasiveness, etc.

In an embodiment, the method comprises exposing the cancer cells to a cancer treatment while culturing the cancer cells in the selected PDS. The method also comprises, in this embodiment, determining a response of the cancer cells to the cancer treatment.

In this particular embodiment, the cancer cells cultured in the selected PDS are exposed to a cancer treatment. The cancer treatment applied to the cancer cells cultured in the PDS can be any cancer treatment that mimics a cancer treatment that can be applied in vivo to the subject. For instance, the cancer treatment could be selected from the group consisting of chemotherapy, radiation therapy, immunotherapy, targeted cancer therapy, and any combination thereof.

Chemotherapy is a type of cancer treatment that uses one or more anti-cancer drugs, i.e., chemotherapeutic agents, including, but not limited to, alkylating agents, anthracyclines, cytoskeletal disruptors (taxanes), epothilones, histone deacetylase inhibitors, inhibitors of topoisomerase I, inhibitors of topoisomerase II, kinase inhibitors, nucleotide analogs and precursors thereof, peptide antibiotics, platinum-based agents, retinoids and vinca alkaloids and derivatives thereof. In a particular embodiment, exposing the cancer cells to a cancer treatment comprises adding at least one chemotherapeutic agent to the culture medium, in which the cancer cells are cultured in the PDS.

Radiation therapy, also referred to as radiotherapy, is a cancer treatment comprising irradiating cancer cells using ionizing radiation. In a particular embodiment, exposing the cancer cells to a cancer treatment comprises applying ionizing radiation to the cancer cells cultured in the PDS.

Targeted cancer therapy is a cancer treatment that uses drugs to target specific genes and proteins that are involved in the growth and survival of cancer cells. Targeted therapy can affect the cancer cells themselves, but also the tissue environment that helps a cancer grow and survive, or it can target cells related to cancer growth like blood vessels.

In an embodiment, exposing the cancer cells to a cancer treatment comprises exposing the cancer cells to a cancer immunotherapy while culturing the cancer cells in the selected PDS.

Immunotherapy is a cancer treatment that is based on activating the immune system. Cell-based immunotherapies are effective for some cancers using immune effector cells, such as lymphocytes, macrophages, dendritic cells, natural killer cells, and cytotoxic T lymphocytes to defend the body against cancer by targeting abnormal antigens expressed on the surface of cancer cells. Such immunotherapies also include therapies involving engineered immune cells, such as chimeric antigen receptor (CAR) T cell therapy, and other types of adoptive cell therapies. In these embodiments, exposing the cancer cells to a cancer treatment comprises adding immune effector cells to the cancer cells cultured in the PDS. Other types of immunotherapies include immune checkpoint inhibitors, monoclonal antibodies, oncolytic virus therapy, cancer vaccines and immune system modulators.

In an embodiment, the method comprises analyzing the expression of at least one immunomarker in the cancer cells cultured in the selected PDS prior to exposing the cancer cells to the cancer treatment. The method also comprises analyzing the expression of the at least one immunomarker in the cancer cells cultured in the selected PDS after exposing the cancer cells to the cancer treatment. The method further comprises comparing the expression of the at least one immunomarker in the cancer cells cultured in the selected PDS prior to exposing the cancer cells to the cancer treatment with the expression of the at least immunomarker in the cancer cells cultured in the selected PDS after exposing the cancer cells to the cancer treatment. In this embodiment, the response of the cancer cells to the cancer treatment is determined based on the comparison.

The comparison of expressions of immunomarkers prior to and following cancer treatment can provide valuable diagnostic information of the population of cancer cells surviving the cancer treatment. For instance, the cancer treatment may cause a change in the populations of cancer cells cultured in the PDS, such as by mainly killing a highly proliferative population of cancer cells thereby leaving a more quiescent, dormant population of cancer cell remaining viable following the cancer treatment. The comparison in expression of immunomarkers can thereby give, among others, such information relating to any changes in cancer cell populations and giving information of which cancer cell populations that are mainly affected by the cancer treatment and the characteristics of the cancer cells escaping or surviving the cancer treatment.

In an embodiment, the method comprises determining the viability of the cancer cells cultured in the selected PDS prior to exposing the cancer cells to the cancer treatment. The method also comprises determining the viability of the cancer cells cultured in the selected PDS after exposing the cancer cells to the cancer treatment. The method further comprises comparing the viability of the cancer cells cultured in the selected PDS prior to exposing the cancer cells to the cancer treatment with the viability of the cancer cells cultured in the selected PDS after exposing the cancer cells to the cancer treatment. The method additionally comprises determining the response of the cancer cells to the cancer treatment based on the comparison.

Viability as used herein is a measure of the proportion of live cells within a cell population. Cell viability may be assayed through measurement of, for instance, metabolic activity, adenosine triphosphate (ATP) count or cell proliferation.

In this embodiment, the in vitro cancer model is thereby used to monitor and assess the efficacy of the cancer treatment and where this information is of high value as a prediction of the efficacy of the cancer treatment when applied to the subject. Thus, if the cell viability is comparatively lower following the cancer treatment, the cancer treatment is determined to be effective, whereas if the cell viability after the cancer treatment is not significantly different from the cell viability prior to the cancer treatment, the cancer treatment is determined to be less effective for the subject and another cancer treatment should instead be investigated.

In an embodiment, the method comprises determining at least one cancer cell property of viable cancer cells after exposing the cancer cells to the cancer treatment. In this embodiment, the cancer cell property is preferably selected from the group consisting of cancer stem cell-ness, proliferation, migration, apoptosis, epithelial-to-mesenchymal transition, therapy resistance, cell metabolism, cell-to-cell communication, and differentiation. The method then comprises determining the response of the cancer cells to the cancer treatment based on the at least one determined cancer cell property.

In this embodiment, properties of viable cancer cells surviving the cancer treatment are determined. These cell properties provide valuable information of characteristics of cancer cells likely to survive the cancer treatment as when applied to the subject. For instance, the determined cell properties could indicate whether the cancer cells are highly differentiated or more cancer stem cell-ness. Also information relating to proliferation and the capability of the cancer cells to migrate from the tumor and their invasiveness may be valuable to assess the potential of the cancer cells for forming metastases in the subject.

In an embodiment, the at least one cancer cell property is determined by analyzing the expression of at least one cancer cell property marker in viable cancer cells after exposing the cancer cells to the cancer treatment. This embodiment also comprises determining the at least one cancer cell property based on the expression of the least one cancer cell property marker.

Thus, the expression of one or more cell property markers are analyzed and used to determine the at least one cancer cell property. For instance, gene expression associated with cancer stem cell-ness, proliferation, migration or differentiation could be analyzed as previously described herein or the analysis can be done on protein levels, such as using immunoassays. Cancer cell property markers associated with cancer stem cell-ness, proliferation, migration and differentiation are well known in the art.

The in vitro model of the invention can also be used to find new immunotargets that could be useful as target for cancer therapy. Hence, an aspect of the invention relates to a method for identifying an immunotarget for cancer therapy for a subject, see FIG. 15. The method comprises culturing, in step S10, cancer cells in a PDS obtained from a tumor in the subject. The method also comprises analyzing, in step S11, expressions of immunomarkers in the cancer cells. The method further comprises selecting, in step S12, at least one immunotarget for cancer therapy for the subject based on the expressions of immunomarkers in the cancer cells.

Thus, the in vitro model could be used to analyze the expression of immunomarkers in the cancer cells in the PDS and where this expression of immunomarkers is modulated by the microenvironment of the PDS. The various immunomarkers expressed by the cultured cancer cells could then be assessed to find potential new immunotargets for cancer therapy. For instance, a PDS obtained from a given subject may significantly induce expression of a particular immunomarker in the cancer cells when cultured in the PDS. This immunomarker may then be selected as a potential target for immunotherapy, such as by using a targeted cancer therapy affecting cells expressing the immunomarker, for instance, by using antibodies that specifically binds to the immunomarker. Hence, the in vitro model could be used for determining individualized or personalized cancer therapy by identifying immunotargets that are suitable for usage in a particular subject.

The in vitro model could also be used to find general or common immunotargets by using PDSs from a plurality of different subjects in order to find one or more immunomarkers that are expressed in the cancer cells in all, or at least a minimum portion of the PDSs. Such an immunomarker can then be suitable as immunotarget in general, i.e., not necessarily subject-specific.

In an embodiment, the cancer cells cultured in the PDS in step S10 are not obtained from the tumor, from which the PDS is obtained.

In an embodiment, the method also comprises adding the cancer cells to the PDS.

In an embodiment, the expression of immunomarkers is analyzed in step S11 by analyzing, in the cancer cells, the expressions of genes coding for proteins involved in immunoregulatory and/or inflammatory processes. Instead of, or as a complement, to performing the analysis on gene level, such as using qPCR or gene expression array, the analysis could be performed by analyzing proteins in the cancer cells and/or in the culture medium, such as using immunoassays.

Generally, a wide distribution in the expression of a gene in cancer cells cultured in different PDSs generally indicates that the microenvironment of the PDSs is capable of influencing the expression of the gene in the cancer cells as cultured in the different PDSs. Such a gene having a wide distribution of gene expression is generally a good candidate as immunomarker as used herein.

In an embodiment, the method comprises analyzing, in step S11, the expressions of the immunomarkers in the cancer cells cultured in multiple PDSs obtained from tumors in multiple subjects.

In such an embodiment, step S12 comprises selecting at least one immunotarget for cancer therapy for the subject based on the distribution of the expressions of immunomarkers in the cancer cells cultured in the multiple PDSs.

In a particular embodiment, step S11 also comprises analyzing an association between the expressions of the immunomarkers in the cancer cells cultured in multiple PDSs with one or multiple tumor properties or clinical variables. In such a particular embodiment, step S12 comprises selecting at least one immunotarget for cancer therapy for the subject based on the distribution of the expressions of immunomarkers in the cancer cells cultured in the multiple PDSs and based on any association of immunomarker expression with tumor properties (clinical variables).

In an embodiment, the method comprises culturing cancer cells in 2D culture and analyzing the expressions of the immunomarkers in the cancer cells cultured in the 2D culture. In this embodiment, the method also comprises comparing the expressions of the immunomarkers in the cancer cells cultured in the PDS with the expressions of the immunomarkers in the cancer cells cultured in the 2D culture. The at least one immunotarget for cancer therapy for the subject is then selected based on the comparison.

Instead of, or as a complement to, using a 2D culture as reference an artificial 3D matrix, i.e., non-patient-derived scaffold, such as matrigel or chitosan/alginate scaffold, could be used as reference by culturing cancer cells in such 3D culture and analyzing the expressions of the immunomarkers in the cancer cells cultured in the 3D culture.

In either case, the comparison provides information of the capability of the PDS to modulate the expression of immunomarkers in the cancer cells when cultured on the PDS. Hence, by comparing the expression of immunomarkers from the cancer cells when cultured in the PDS with the expression of immunomarkers from the cancer cells in the 2D or 3D culture any significant change in expression of immunomarkers, such as upregulation or downregulation, is a direct consequence of the microenvironment of the PDS and thereby provides valuable information of the tumor in the subject.

In a particular embodiment, the method comprises selecting, in step S12, at least one immunotarget to be at least one of the immunomarkers that is significantly differently expressed in the cancer cells cultured in the PDS as compared to in the cancer cells cultured in the 2D or 3D culture.

In an embodiment, the method also comprises comparing the expressions of the immunomarkers with control levels of expressions of immunomarkers. In such an embodiment, the method comprises selecting, in step S12, the at least one immunotarget for cancer therapy for the subject based on the comparison.

In an embodiment, the control level is the level of expression from cancer cells as cultured in various different PDSs. In this embodiment, the method comprises analyzing the expressions of the immunomarkers in the cancer cells cultured in multiple PDS obtained from tumors in multiple subjects. The method also comprises determining the control levels of expressions of immunomarkers based on the expressions of the immunomarkers in the cancer cells cultured in the multiple PDSs.

For instance, average levels of expressions of the immunomarkers can be determined for the cancer cells cultured in these multiple control PDSs. Such average levels could then be used as control levels to which the immunomarker expressions in culture cells cultured in the PDS obtained from a given subject is compared. The comparison can thereby be used to identify subject or tumor specific properties of the PDS that can be used when selecting a cancer therapy that is suitable for the specific properties as determined for the PDS.

In an embodiment, the immunomarkers are selected from the group consisting of PDL1, CSF1, CCL2, PDL2 and major histocompatibility complex class I-related gene protein (MR1). Other examples of immunomarkers include CD80, also referred to as B7-1, and CD86, also referred to as B7-2.

A related aspect of the invention defines a method of determining a tumor-specific immunomarker, see FIG. 16. The method comprises culturing, in step S20, cancer cells in a PDS obtained from a tumor in the subject. The method also comprises analyzing, in step S21, expressions of immunomarkers in the cancer cells. The method further comprises comparing, in step S22, the expressions of the immunomarkers with control levels of expressions of immunomarkers and determining, in step S23, the tumor-specific immunomarker for the tumor in the subject based on the comparison.

In an embodiment, the cancer cells cultured in the PDS in step S20 are not obtained from the tumor, from which the PDS is obtained.

In an embodiment, the method also comprises adding the cancer cells to the PDS.

In an embodiment, the method comprises analyzing, in step S21, the expressions of the immunomarkers in the cancer cells cultured in multiple PDSs obtained from tumors in multiple subjects and determining, in step S23, the control levels of expressions of immunomarkers based on the expressions of the immunomarkers in the cancer cells cultured in the multiple PDSs. The control levels could, for instance, be determined as average or median levels of expressions of immunomarkers in the multiple PDSs.

In an embodiment, determining the tumor-specific immunomarker in step S23 comprises selecting a tumor-specific immunomarker for the tumor in the subject that is significantly differently expressed in the cancer cells cultured in the PDS from the tumor as compared to the control levels of expressions of immunomarkers.

The determined tumor-specific immunomarker can then be selected as a target for immunotherapy for the tumor.

In another embodiment of a method of determining a tumor-specific immunomarker, the method comprises culturing cancer cells in multiple PDSs obtained from multiple tumors from different subjects. The method also comprises analyzing gene expressions of multiple genes in the cancer cells. The method further comprises comparing levels of the gene expressions from the cancer cells cultured in the multiple PDSs and selecting at least one gene among the multiple genes based on the distribution of the levels of the gene expressions in the cancer cells cultured in the multiple PDSs.

In a particular embodiment, selecting the at least one gene comprises selecting at least one gene having a wide distribution in the levels of the gene expressions in the cancer cells cultured in different PDSs.

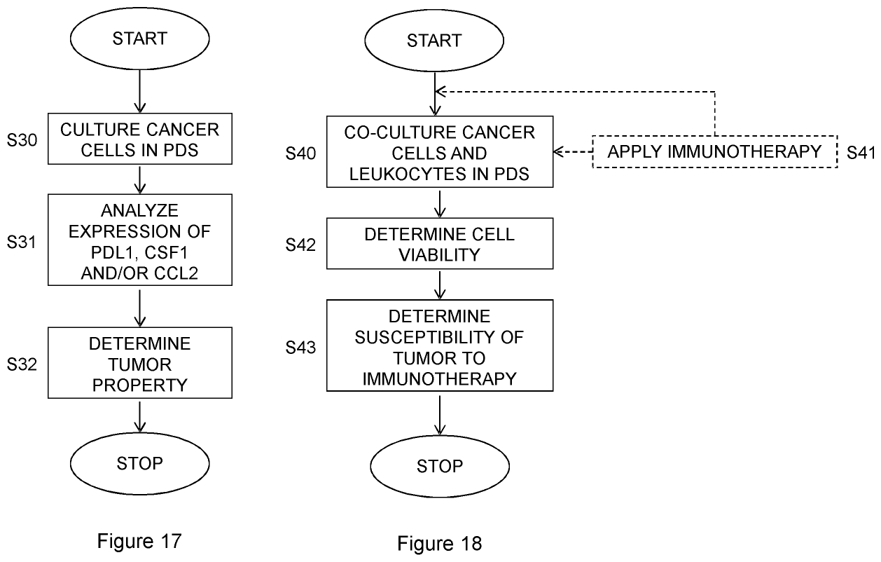

A particular aspect of the invention relates to a method of determining at least one tumor property of a tumor in a subject, see FIG. 17. The method comprises culturing, in step S30, cancer cells, preferably breast cancer cells, in a PDS obtained from a tumor in the subject. The method also comprises analyzing, in step S31, the expression of at least one immunomarker selected from the group consisting of PDL1, CSF1 and CCL2 in the cancer cells. The method further comprises determining, in step S32, the at least one tumor property based on the expression of the at least one immunomarker.

In an embodiment, the cancer cells cultured in the PDS in step S30 are not obtained from the tumor, from which the PDS is obtained.

In an embodiment, the method also comprises adding the cancer cells to the PDS.

Experimental data as presented herein shows that there is a correlation between the expression of PDL1, CSF1 and CCL2 and tumor properties. Hence, by analyzing the expression of at least one of these immunomarkers in step S31 valuable information about the tumor in the subject and from which the PDS is derived can be obtained.

In a particular embodiment, the expression of PDL1 in the cancer cells is analyzed in step S31. In such an embodiment, the method comprises determining, in step S32, at least one tumor property selected from the group consisting of tumor malignancy grade, ductal vs. lobular carcinoma, and disease-free survival (DFS) period based on the expression of PDL1.

DFS as used herein indicates as a period of time without recurrences or till death by cancer.

Thus, the expression of the immunomarker PDL1 could be used to classify the grade of the tumor as the expression of the gene PDL1 was significantly higher in cancer cells cultured in PDSs obtained from high grade tumors (Grade 3) as compared to cancer cells cultured in PDSs obtained from low grade tumors (Grade 1 and 2). Furthermore, PDL1 gene expression was also higher in cancer cells cultured in PDSs from ductal carcinoma as compared to when cultured in PDSs from lobular carcinoma. Moreover, PDSs from patients with shorter times without relapses or tumor progression showed a trend to induce higher levels of PDL1 in cancer cells

In another particular embodiment, the expression of CSF1 in the cancer cells is analyzed in step S31. In such an embodiment, the method comprises determining, in step S32, at least one tumor property selected from the group consisting of estrogen receptor (ER) positive versus ER negative tumor and DFS period based on the expression of CSF1.

Experimental data indicates that higher levels of CSF1 were induced by PDSs from subjects with shorter times without relapses or tumor progression as compared to PDSs from subjects longer DFS periods.

In a further particular embodiment, the expression of CCL2 in the cancer cells is analyzed in step S31. In such an embodiment, the method comprises determining, in step S32, at least one tumor property selected from the group consisting of tumor recurrence based on the expression of CCL2.

PDSs inducing high CCL2 gene expression in cancer cells were generally obtained from patients that had significantly fewer recurrences and longer time without relapses.

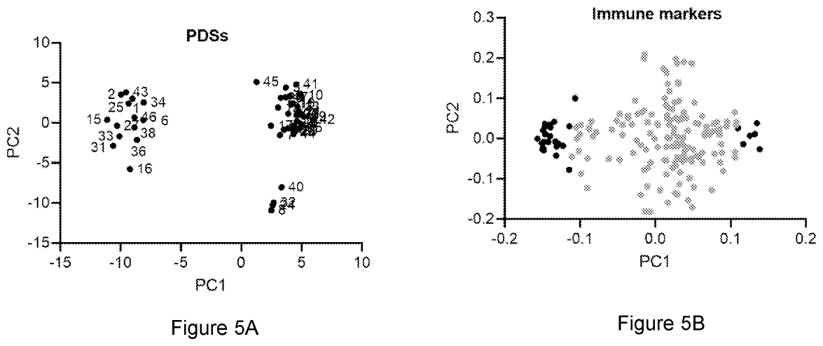

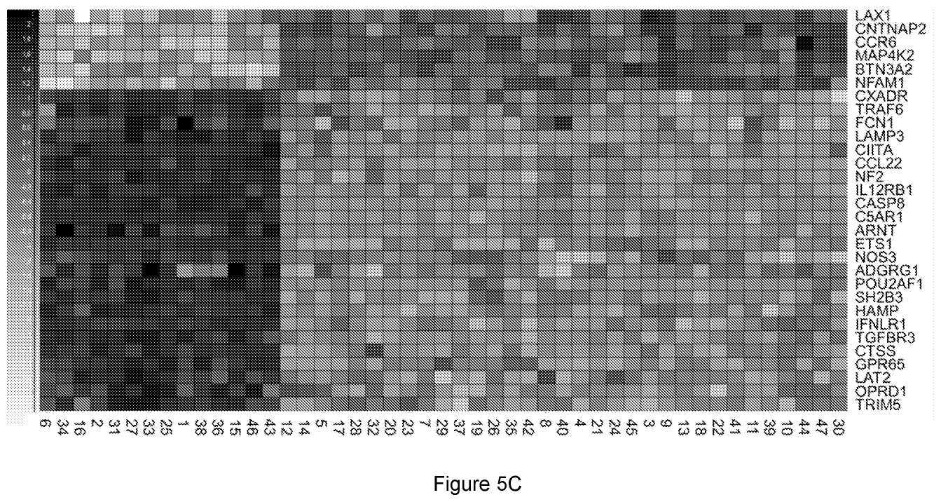

Examples of other immunomarkers that could be analyzed in step S31 in the cancer cells cultured in the PDS in step S30 can be selected among the immunomarkers listed in Table 3 showing statistics of the association of immunomarker expression with tumor properties (clinical variables), such as tumor recurrence, tumor grade, ER status, ER status and lymph node metastasis. Hence, also those immunomarkers listed in Table 3 statistically associated with at least one tumor property could be used according to the invention. Reference is also made to FIG. 5C that is a heat map showing the expression of 30 selected immunomarkers along different PDSs using average linkage as clustering method and Euclidean distances as the distance measure. In a particular embodiment, the immunomarkers shown in FIG. 5C could be used according to the invention.

Association between a specific gene and clinical variables is important to identify suitable immunomarkers. However, a gene having a wide range of gene expression in cancer cells cultured in different PDSs, i.e., high expression of the gene in cancer cells cultured in some PDSs and low expression of the gene in cancer cells cultured in other PDSs, is, as mentioned in the foregoing, more relevant when identifying an immunomarker.

FIGS. 5A to 5C illustrate that multiple PDSs from different patients are clustered, in this particular case, into two separate clusters of PDSs based on the immunomarker gene expression. It is also clear from these figures that some particular immunomarkers are more relevant in terms of characterizing the microenvironment of PDSs than others. Hence, the analysis of immunomarker gene expression in cancer cells cultured in PDSs from different tumors and patients and clustering the PDSs based on the immunomarker gene expression can be used to define the properties of the microenvironment in a PDS or a group of PDS, such as immune activating properties of the microenvironment.

The cancer cells may be co-cultured with other cells, such as leukocytes, in the PDS. Such co-cultures may be of diagnostic and clinical relevance in relation to immunotherapy of the cancer disease.

An aspect of the invention thereby relates to a method of determining susceptibility of a tumor to immunotherapy, see FIG. 18. The method comprises co-culturing, in step S40, cancer cells and leukocytes in a PDS obtained from a tumor in a subject. The method also comprises determining, in step S42, the viability of the cancer cells and determining, in step S43, the susceptibility of the tumor in the subject to immunotherapy based on the determined viability of the cancer cells.

In an embodiment, the leukocytes co-cultured with the cancer cells represent the immunotherapy. In another embodiment, another, or additional, immunotherapy may be applied in the optional step S41. This step S41 could be performed prior to and/or during the co-culturing step S40. For instance, the leukocytes co-cultured with the cancer cells in S40 could be pre-treated, such by addition of immune checkpoint inhibitors, monoclonal antibodies, and/or immune system modulators prior to adding the leukocytes to the cancer cells. Alternatively, or in addition, the cancer cells could be pre-treated prior to co-culturing with the leukocytes. It is also or instead possible to apply the immunotherapy onto the cancer cells and leukocytes during at least a portion of the co-culturing in step S40.

In an embodiment, the cancer cells co-cultured in the PDS in step S40 are not obtained from the tumor, from which the PDS is obtained.

In an embodiment, the method also comprises adding, either simultaneously or sequentially, the cancer cells and the leukocytes to the PDS.

Hence, this method can be used to predict the susceptibility of the tumor to immunotherapy by determining the cell viability of the cancer cells in the co-culture to thereby get an indication of the effectiveness of the immunotherapy, such as represented by the leukocytes or the immunotherapy applied in step S41, in killing cancer cells in the particular PDS. Cancer cells co-cultured in different PDSs may be more or less vulnerable to immunotherapy depending on the particular microenvironment of the PDS. For instance, an effective killing of cancer cells by the leukocytes, i.e., low cell viability of the cancer cells, in the co-culture in the PDS indicates that immunotherapy may be a suitable cancer therapy strategy for eradicating the tumor, from which the PDS is obtained. Correspondingly, high cell viability indicates that the particular microenvironment of the PDS protects the cancer cells and thereby makes them less vulnerable to immunotherapy.

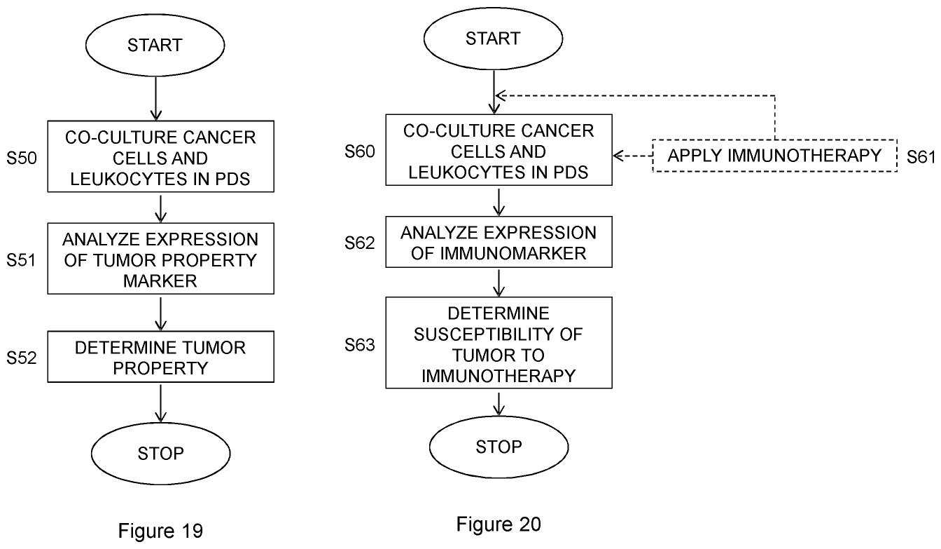

Another aspect of the invention relates to a method of determining at least one tumor property of a tumor in a subject, see FIG. 19. The method comprises co-culturing, in step S50, cancer cells and leukocytes in a PDS obtained from a tumor in a subject. The method also comprises analyzing, in step S51, the expression of at least one tumor property marker in the viable cancer cells and determining, in step S52, at least one tumor property based on the expression of the at least one tumor property marker in the viable cancer cells co-cultured with the leukocytes in the cell-free scaffold.

In an embodiment, the cancer cells co-cultured in the PDS in step S50 are not obtained from the tumor, from which the PDS is obtained.

In an embodiment, the method also comprises adding, either simultaneously or sequentially, the cancer cells and the leukocytes to the PDS.

Thus, the expression of at least one tumor property marker in the cancer cells that survive the co-culturing with the leukocytes in the PDS is analyzed in step S51 and used in step S52 to determine at least one tumor property of the tumor in the subject, form which the PDS is derived.

The tumor property of the tumor can be selected among the previously exemplified tumor properties including tumor malignancy grade, cancer type, tumor location, differentiation capability, proliferation capability, infiltration capability, metastasizing capability, EMT capability and CSC capability.

A tumor property marker is then correlated with a tumor property in terms of a significant upregulation or downregulation of the transcription of the gene encoding the tumor property marker and/or a significant increase or decrease in the amount of the tumor property marker is correlated with the given tumor property. For instance, co-culturing cancer cells with leukocytes increases the expression of gene encoding PDL1 in the cancer cells surviving the co-culturing in the PDS. PDL1 is in turn correlated with, among others, tumor malignancy grade and DFS period.

In an embodiment, the method further comprises determining the susceptibility of the tumor in the subject to cancer treatment based on the determined at least one tumor property. Thus, information of the at least one tumor property as obtained by analysis of the expression of the at least one tumor property marker can be used to determine the susceptibility of the tumor in the subject to a cancer treatment, such as an immunotherapy. This means that the information can be used in selecting a suitable cancer treatment to be applied to the subject.

A particular aspect of the invention relates to a method of determining susceptibility of a tumor to immunotherapy, see FIG. 20. The method comprises co-culturing, in step S60, cancer cells and leukocytes in a PDS obtained from a tumor in a subject. The method also comprises analyzing, in step S62, the expression of at least one immunomarker in the cancer cells from the co-culture of cancer cells and leukocytes in the PDS. The method further comprises determining, in step S63, the susceptibility of the tumor in the subject to immunotherapy based on the expression of the at least one immunomarker.

In an embodiment, the leukocytes co-cultured with the cancer cells represent the immunotherapy. In another embodiment, another, or additional, immunotherapy may be applied in the optional step S61. This step S61 could be performed prior to and/or during the co-culturing step S60. For instance, the leukocytes co-cultured with the cancer cells in S60 could be pre-treated, such by addition of immune checkpoint inhibitors, monoclonal antibodies, and/or immune system modulators prior to adding the leukocytes to the cancer cells. Alternatively, or in addition, the cancer cells could be pre-treated prior to co-culturing with the leukocytes. It is also or instead possible to apply the immunotherapy onto the cancer cells and leukocytes during at least a portion of the co-culturing in step S60.

In an embodiment, the cancer cells co-cultured in the PDS in step S60 are not obtained from the tumor, from which the PDS is obtained.

In an embodiment, the method also comprises adding, either simultaneously or sequentially, the cancer cells and the leukocytes to the PDS.

In an embodiment, the method comprises culturing cancer cells in a PDS obtained from a tumor in the subject. The method also comprises analyzing the expression of the at least one immunomarker in the cancer cells from the culture of cancer cells in the PDS. The method further comprises comparing the expression of the at least one immunomarker in the cancer cells from the culture with the expression of the at least one immunomarker in the cancer cells from the co-culture. In this embodiment, the method comprises determining, in step S63, the susceptibility of the tumor in the subject to immunotherapy based on the comparison.

Thus, in this particular embodiment, a reference or control is used in terms of a culture of cancer cells in the PDS without any leukocytes and the expression of the at least one immunomarker is analyzed in the cancer cells cultured in the PDS without any leukocytes. This reference or control thereby allows assessing the influence or effect of the leukocytes in the co-culture to the expression of the at least one immunomarker in the cancer cells.

Another example of reference or control is a co-culture of cancer cells and leukocytes in a traditional 2D or 3D culture as previously disclosed herein. Such a reference or control allows assessing the influence or effect of the microenvironment of the PDS to the expression of the at least one immunomarker in the cancer cells co-cultured with the leukocytes.

In an embodiment, the method also comprises determining at least one tumor property of viable cancer cells co-cultured with the leukocytes in the PDS. In this embodiment, the susceptibility of the tumor in the subject to immunotherapy is determined in step S63 based on the expression of the at least one immunomarker and based on the at least one tumor property.

In a particular embodiment, the method comprises analyzing the expression of PDL1 in the cancer cells from the co-culture of cancer cells and leukocytes in the cell-free scaffold in step S62. In such a case, the susceptibility of the tumor in the subject to immunotherapy is determined in step S63 based on the expression of PDL1.

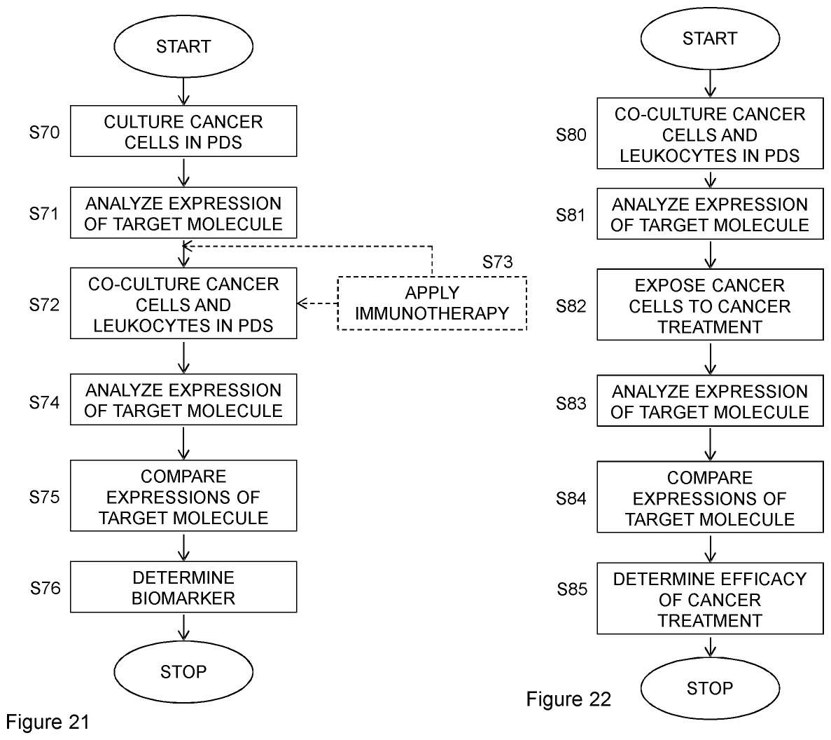

The co-culturing of cancer cells and leukocytes in the PDS can also be used to identify biomarkers that can be used to predict susceptibility of a tumor to immunotherapy. Hence, the invention also relates to a method of determining a biomarker for susceptibility of a tumor to immunotherapy, see FIG. 21. The method comprises culturing, in step S70, cancer cells in a PDS obtained from a tumor in a subject. The method also comprises analyzing, in step S71, the expression of at least one target molecule, such as at least one immunomarker, in viable cancer cells from the culture of cancer cells in the PDS. The method further comprises co-culturing, in step S72, cancer cells and leukocytes in a PDS obtained from a tumor in the subject and analyzing, in step S74, the expression of the at least one target molecule, such as the at least one immunomarker, in viable cancer cells from the co-culture of cancer cells and leukocytes in the PDS. The method additionally comprises comparing, in step S75, the expression of the at least one target molecule, such as the at least one immunomarker, in the viable cancer cells from the culture with the expression of the at least one target molecule, such as the at least one immunomarker, in the viable cancer cells from the co-culture and determining, in step S76 and among the at least one target molecule, such as among the at least one immunomarker, a biomarker for susceptibility of the tumor to immunotherapy based on the comparison.

In an embodiment, the leukocytes co-cultured with the cancer cells represent the immunotherapy. In another embodiment, another, or additional, immunotherapy may be applied in the optional step S73. This step S73 could be performed prior to and/or during the co-culturing step S72. For instance, the leukocytes co-cultured with the cancer cells in S72 could be pre-treated, such by addition of immune checkpoint inhibitors, monoclonal antibodies, and/or immune system modulators prior to adding the leukocytes to the cancer cells. Alternatively, or in addition, the cancer cells could be pre-treated prior to co-culturing with the leukocytes. It is also or instead possible to apply the immunotherapy onto the cancer cells and leukocytes during at least a portion of the co-culturing in step S72.

In an embodiment, the cancer cells cultured in the PDS in step S70 and co-cultured in the PDS in step S72 are not obtained from the tumor, from which the PDS is obtained.

In an embodiment, the method also comprises adding the cancer cells to the PDS and adding either simultaneously or sequentially, the cancer cells and the leukocytes to the PDS.

Hence, the expressions of different target molecules, and in particular immunomarkers, by cancer cells are assessed both when culturing the cancer cells alone in a PDS and when co-culturing the cancer cells with leukocytes in a PDS. Any target molecule, such as immunomarker, that is significantly differently expressed in the co-culture as compared to only culturing cancer cells is a potential biomarker for predicting the susceptibility of the tumor to immunotherapy.

Hence, in an embodiment, the method comprises selecting the biomarker to be a target molecule, such as an immunomarker, that is significantly differently expressed in the viable cancer cells from the culture as compared to in the viable cancer cells from the co-culture.

The PDS used in steps S70 and S71 and the PDS used in steps S72 and S74 are derived from a tumor from the same subject. In a particular embodiment, the PDS are derived from the same tumor or tumor sample obtained from the subject. It is also possible to the same PDS in steps S72 and S74 as in step S70 and S71 if the cancer cells are removed from the PDS following step S71.

The co-culturing of cancer cells and leukocytes in a PDS can also be used for determining efficacy of a cancer treatment for a subject having a tumor, see FIG. 22. Such a method comprises a) co-culturing, in step S80, cancer cells and leukocytes in a PDS obtained from a tumor in the subject. The method also comprises b) analyzing, in step S81, the expression of at least one target molecule, such as at least one immunomarker, in the cancer cells. The method further comprises c) exposing, in step S82, the cancer cells to a cancer treatment when co-cultured with leukocytes in the PDS. The method further comprises d) analyzing, in step S83, the expression of the at least one target molecule, such as the at least one immunomarker, in the cancer cells after exposure to the cancer treatment. The method additionally comprises e) comparing, in step S84, the expressions of the at least one target molecule, such as the at least one immunomarker, analyzed in b) and d) and f) determining, in step S85, efficacy of the cancer treatment based on the comparison.

In an embodiment, the cancer cells co-cultured in the PDS in step S80 are not obtained from the tumor, from which the PDS is obtained.

In an embodiment, the method also comprises adding, either simultaneously or sequentially, the cancer cells and the leukocytes to the PDS.

In another embodiment, the method of determining efficacy of a cancer treatment for a subject having a tumor comprises, see FIG. 23, a) co-culturing, in step S90, cancer cells and leukocytes in a PDS obtained from a tumor in the subject before the cancer treatment has been applied to the subject. The method also comprises b) analyzing, in step S91, the expression of at least one target molecule, such as at least one immunomarker, in the cancer cells. The method further comprises c) co-culturing, in step S92, cancer cells and leukocytes in a PDS obtained from a tumor in the subject after the cancer treatment has been applied to the subject. The method additionally comprises d) analyzing, in step S93, the expression of the at least one target molecule, such as the at least one immunomarker, in the cancer cells. The method also comprises e) comparing, in step S94, the expressions of the at least one target molecule, such as at least one immunomarker, analyzed in b) and d) and f) determining, in step S95, efficacy of the cancer treatment based on the comparison.

In an embodiment, the cancer cells co-cultured in the PDS in step S90 are not obtained from the tumor, from which the PDS is obtained.

In an embodiment, the method also comprises adding, either simultaneously or sequentially, the cancer cells and the leukocytes to the PDS.

In the former embodiment, the cancer treatment is applied to the cancer cells (and leukocytes) in co-culture in the PDS and the effect of the cancer treatment on the expressions of at least one target molecule, in particular at least one immunomarker, following versus prior to application of the cancer treatment are analyzed, compared and used to determine the efficacy of the cancer treatment. For instance, a significant reduction in a target molecule, such as immunomarker, correlating with high cell proliferation is an indication that the cancer treatment may be efficient in killing the population of highly proliferating cancer cells.

In the latter embodiment, the effect of the cancer treatment on the microenvironment of the PDS is determined by deriving a (first) PDS from the tumor in the subject prior to cancer treatment and deriving another (second) PDS from the tumor in the subject once the cancer treatment has been applied. The at least one target molecule, such as immunomarker, expressed by the cancer cells co-cultured with the leukocytes can then be used to assess whether the cancer treatment has caused any effect on the microenvironment of the tumor, which in turn affects gene expressions by viable cancer cells, the population of cancer cells surviving the co-culturing with the leukocytes and/or the capability of the leukocytes to target and kill cancer cells in the co-culture.

In a particular embodiment, co-culturing cancer cells as disclosed herein comprises seeding the PDS with the cancer cells and culturing the cancer cells in the PDS for a first period of time. The PDS is then seeded with the leukocytes and the cancer cells and the leukocytes are co-cultured in the PDS for a second period of time.

Hence, it is generally preferred to first seed cancer cells in the PDS and allow the cancer cells to grow therein for a first period of time. This first period of time could, for instance, range from one or more days up to one or more weeks. For instance the first period of time could be about one week, about two weeks, about three weeks, about four weeks or even longer. Then the leukocytes are added to the cancer cells in the PDS and the cancer cells and leukocytes are co-cultured in the PDS for second period of time.

The second period of time could be the same as the first period of time but is generally shorter. For instance, the second period of time could range from one or more hours up to one or more days. As an illustrative example, the second period of time could be 4 hours, 8 hours, 12 hours, 16 hours, 20 hours, 24 hours, 36 hours, 48 hours, 60 hours, 72 hours, 84 hours, 100 hours or even longer.

In an embodiment, the culture medium may optionally be changed when seeding the leukocytes or one more additives may optionally be added to the culture medium in connection with seeding the leukocytes.

In the above described embodiments, a single population of cancer cells, such as a single cancer cell line or a single type of cancer cells, could be cultured in the PDS in step S2 in FIG. 14, step S10 in FIG. 15, step S20 in FIG. 16, step S30 in FIG. 17 and/or step S70 in FIG. 21 or co-cultured in the PDS together with leukocytes in step S40 in FIG. 18, step S50 in FIG. 19, step S60 in Figure step S72 in FIG. 21, step S80 in FIG. 22 and/or steps S90 and S92 in FIG. 23. Alternatively, multiple populations of cancer cells could be cultured in the PDS or co-cultured in the PDS together with leukocytes.

Correspondingly, a single population of leukocytes or multiple populations of leukocytes could be co-cultured with the cancer cells in step S40 in FIG. 18, step S50 in FIG. 19, step S60 in FIG. 20, step S72 in FIG. 21, step S80 in FIG. 22 and/or steps S90 and S92 in FIG. 23. Multiple leukocytes as referred to herein include, as is further described herein, a combination of lymphocytes and macrophages and/or monocytes, a combination of different lymphocytes, such as a combination of natural killer (NK) cells, T cells and/or B cells, a combination of different T cells, different B cells and/or different NK cells, a combination of macrophages and monocytes, or a combination of different macrophages and/or different monocytes.

In the case of co-culturing in step S40 in FIG. 18, step S50 in FIG. 19, step S60 in FIG. 20, step S72 in FIG. 21, step S80 in FIG. 22 and/or steps S90 and S92 in FIG. 23, a single population of cancer cells could be co-cultured with a single population of leukocytes, multiple populations of cancer cells could be co-cultured with a single population of leukocytes, a single population of cancer cells could be co-cultured with multiple populations of leukocytes or multiple populations of cancer cells could be co-cultured with multiple populations of leukocytes.

In the above described embodiments, the expression of a single immunomarker or a single target molecule is analyzed and optionally selected in step S11 in FIG. 15, step S21 in FIG. 16, step S62 in FIG. 20, steps S71 and S74 in FIG. 21, steps S81 and S83 in FIG. 22 and/or steps S91 and S93 in FIG. 22. Alternatively, the expressions of multiple immunomarkers or target molecules are analyzed and optionally selected in any of the above described method steps.

The invention also relates to a tumor scaffold comprising a PDS obtained from a tumor in a subject, cancer cells cultured in the PDS and leukocytes co-cultured with the cancer cells in the PDS.

In an embodiment, the cancer cells cultured in the PDS are not obtained from the tumor, from which the PDS is obtained.

In an embodiment, the leukocytes comprise, or are, lymphocytes, i.e., natural killer (NK) cells, T cells and/or B cells. In a particular embodiment, the leukocytes are T cells. The T cells could be non-activated T cells but are preferably activated T cells. For instance, the T cells could be activated by anti-CD3 antibodies and/or anti-CD28 antibodies. In addition, or alternatively, various T cell activators, including interleukins (ILs), such as IL2 could be used to activate the T cells.

In another embodiment, the leukocytes comprise, or are, macrophages and/or monocytes. For instance, monocytes could be used as leukocytes to monitor, in the co-culture with cancer cells in the PDS, their differentiation into macrophages. In such a case, the type of macrophages that the monocytes differentiate into, such as classically-activated (M1) macrophages, alternatively-activated (M2) macrophages or regulatory macrophages (Mregs), could provide valuable information of the effect of the microenvironment of the PDS and thereby of the tumor, from which it is derived, on monocyte differentiation.

It is also possible to use a combination of various leukocytes including neutrophils, eosinophils, basophils, lymphocytes and monocytes in the co-culture with cancer cells in the PDS and, in particular a combination of lymphocytes and monocytes, such as a combination of T cells and monocytes.

In an embodiment, the cancer cells used in the culture or co-culture in the PDS are preferably selected from the group consisting of breast cancer cells, colorectal cancer and colon cancer cells. In a particular embodiment, the cancer cells are breast cancer cells, such as estrogen receptor (ER) positive breast cancer cells, and more preferably MCF7 breast cancer cells.

The cancer cells cultured or co-cultured in the PDS according to any of the embodiments described above in connection with FIGS. 15 to 23 could be selected as described herein in connection with FIG. 14.

### EXAMPLES

### Example 1—an In Vitro Cancer Model Based on Patient-Derived Tumor Tissue—Breast Cancer

Provided are details of the preparation of an in vitro cancer model based on decellularized patient-derived breast cancer tumor scaffolds repopulated with breast cancer cells. Provided are also further details of the analysis of the PDS microenvironment and the expression of specific immune-related molecules, which are maintained in the tumor microenvironment even after decellularization.

Patient material. The use of patient material for this project was approved by the Regional Research Ethics Committee in Gothenburg (DNR: 515-12 and T972-18). All research was performed according to ethical guidelines and informed consent was obtained from all the participants. 101 frozen tumors from the Breast Cancer Biobank (Pre- and postmenopausal women, diagnosis 1980-1999), and 12 fresh breast cancer primary samples after surgery (collected directly after surgery from the Clinical Pathology Diagnostic Unit at the Sahlgrenska University Hospital, Gothenburg, Sweden) were used.

Tumor decellularization and generation of patient-derived-scaffolds (PDSs). Breast tumor samples were decellularized as previously described in Landberg et al. 2020. In brief, breast tumor samples were washed twice with lysis buffer (0.1% SDS, 0.02% Na-Azide, 5 mM 2H₂O—Na₂-EDTA, 0.4 mM PMSF; Sigma-Aldrich) for 6 hours followed by a rinse step in the same buffer without SDS for an additional 15 min. Then, decellularized tumors were washed for 72 hours in distilled water, which was renewed every 12 hours, and with a last 24-hour wash in PBS (Medicago). After decellularization, PDSs were cut in 6 mm diameter pieces using a biopsy punch needle and sliced in 150 μm slices using a CM3050-S cryotome (Leica) to get several slices from the same PDS. PDS slices were sterilized in peracetic acid % (Sigma-Aldrich), 1 hour at room temperature (20-25° C.), followed by 24 hours wash in PBS with 1% Antibiotic-Antimycotic (Thermo Fisher Scientific), at 37° C. and gentle agitation (175 rpm; Incu-Shaker™ 10 L, Benchmark). PDSs were then stored at 4° C. in storage buffer (PBS, 0.02% Na-Azide, 5 mM EDTA) until use.

Protein composition analysis of patient-derived scaffolds by mass spectrometry. Preservation of specific molecules in the decellularized patient-derived scaffold was verified using mass spectrometry after decellularization. The protein composition of 45 breast tumor PDSs was analyzed. 3315 unique proteins were detected, and the Pathway Enriched Analysis revealed several categories of proteins associated with the innate and adaptive immunity. These immune and immunity pathways were found within the top 20 enriched pathways present as enriched proteins within the PDS composition (Table 1).

Cell lines and culture conditions. MCF7 (American Type Culture Collection, ATCC; Luminal A subtype) and MDA-MB-231 (ATCC; Triple Negative B subtype) breast cancer cell lines were cultured in DMEM medium, 10% fetal bovine serum, 1% penicillin/streptomycin, 1% L-glutamine (Thermo Fisher Scientific) and 1% MEM Non-Essential Amino Acids (Sigma-Aldrich), or RPMI-1640 medium, 10% fetal bovine serum, 1% penicillin/streptomycin, 1% sodium pyruvate and 1% L-glutamine (Thermo Fisher Scientific), respectively. The MCF7/Luciferase (MCF7/Luc) (GenTarget Inc; Luminal A subtype) breast cancer cell line was cultured in RPMI-1640 (1% penicillin/streptomycin, 1% sodium pyruvate and 1% L-glutamine) and 10% of heat-inactivated bovine serum. Cells were cultured at 37° C. in a 5% CO₂ humidified atmosphere, and cell media was renewed every 3-4 days. For passaging or before addition to PDSs, adherent cultures were detached with Trypsin, centrifuged 3 min at 150×g rpm and re-suspended in fresh media to eliminate Trypsin traces.

Patient-derived scaffold re-cellularization. Prior to re-cellularization, PDSs were soaked overnight in PBS with 1% Antibiotic-Antimycotic, after which they were placed in cell culture media for 1 hour to remove residual storage buffer. PDS slices were then placed in a 48 wells-plate and seeded with 3×cells in 500 μl of cell line specific media supplemented with 1% Antibiotic-Antimycotic. After 24 h, the PDSs were transferred to a new plate containing fresh culture media. This process was repeated once or twice a week, up to 21 days of cultivation.

### Example 2—PDS Influence the Cancer Specific Expression of Immunotargets for Cancer Therapy

Gene expression analysis of immune markers in cancer cells growing in PDSs. Certain genes in cancer cells may modulate their likelihood to respond to various immunotherapies. In order to establish if the microenvironment of different PDSs can trigger changes in the expression of such immunotherapy-related target genes in cancer cells, the expression of six relevant genes encoding proteins involved in immunoregulatory and inflammatory processes, CCL2, CSF1, CD47, MR1, PDL1 (CD274), and PDL2, were analyzed. CCL2 and CSF1 genes encode two cytokines secreted by cancer cells and these cytokines are involved in the recruitment of monocytes from the blood vessels; moreover, CSF1 plays an essential role in the regulation of survival, proliferation and differentiation of macrophages and monocytes. CD47, PDL1 and PDL2 genes encode surface proteins that enable cancer cells to evade the action of the immune system: CD47 molecule is a potent “do not eat me” signal inhibiting the phagocytosis by macrophages, PDL1 and PDL2 are ligands that binds its receptor expressed by the T lymphocytes (T cells) (PD1) and inhibits T cell activation and cytokine production. MR1 gene codes a major histocompatibility complex class I-related protein, which it can be recognized by specific subgroup of T cells and it has been associated to the susceptibility of the cancer cells to be killed by the T cells.

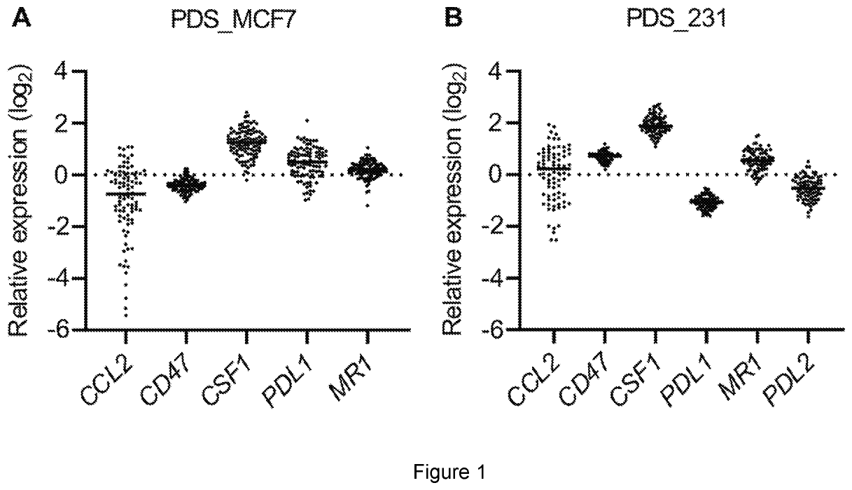

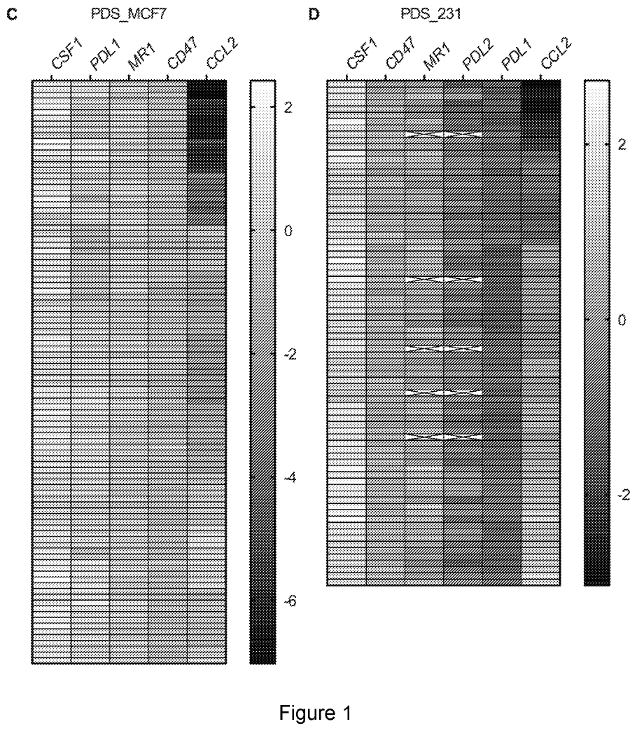

For this study, two well-established breast cancer cells lines representing two different subtypes were used; the luminal A MCF7 cell line, and the triple negative MDA-MB-231 cell line. 101 PDSs originating from frozen biobanked tumors with available clinical follow-up data, were repopulated with MCF7 cells and, 85 PDSs were repopulated with MDA-MB-231 cells. After 21 days of culture in PDS, gene expression was analyzed by qPCR and normalized to corresponding gene expression from a pool of several 2D passages of the same types of cells cultured (FIG. 1).

The gene expression in cancer cells was analyzed as follows. RNA extraction from cancer cells was performed in RTL buffer (Qiagen) and using the RNeasy Micro Kit including DNase treatment in a QIAcube machine (Qiagen), following the manufacturers instructions. When the entire PDSs was used, samples were homogenized using a stainless-steel bead in TissueLyzer II (Qiagen) for 2×5 min at 25 Hz, and centrifuged at full speed for 3 min. RNA concentration was measured by NanoDrop (Thermo Fisher Scientific). Complementary DNA synthesis was carried out with a GrandScript cDNA synthesis kit (TATAA Biocenter) in a T100 Thermal Cycler (BioRad) in 20 μl reaction mix at 25° C. for 5 min, 42° C. for 30 min, 85° C. for 5 min followed by cooling to 4° C. until subsequent analysis. RNA Spike II (TATAA Biocenter) was previously added to every sample as an RNA stability control. Quantitative PCR (qPCR) was performed on a CFX384 Touch Real-Time PCR Detection System (Bio-Rad) using 1×SYBR

GrandMaster Mix (TATAA Biocenter), 400 nM of each primer and 2 μl diluted cDNA in a final reaction volume of 6 μl. The temperature profile was 95° C. for 2 min followed by 35-50 cycles of amplification at 95° C. for 5 s, 60° C. for 20 s and 70° C. for 20 s and a melting curve analysis at 65° C. to 95° C. with 0.5° C./s increments. Cycle of quantification values were determined by the second derivative maximum method with the CFX Manager Software version 3.1 (Bio-Rad). Data pre-processing was performed using GenEx (MultiD). Gene expression was normalized using reference genes identified with the NormFinder algorithm and expressed as relative quantities (log₂) to control samples. All experiments were conducted in accordance with the Minimum Information for Publication of Quantitative Real-Time PCR Experiments (MIQE) guidelines.

The results from the gene expression analysis (FIG. 1) showed that cancer cells growing in PDSs displayed patient-dependent expression of specific immune markers. Substantial expression variability of the genes of interest between cells growing in different PDSs was observed. In general, the MCF7 cell line was more sensitive to the specific patient-tumor environment, as compared to MDA-MB-231 cells, as indicated by the major spread of the immunomarker expression levels. For both types of breast cancer cell lines however, the PDS-dependent variability was more pronounced for CCL2 showing extensive expression differences between PDSs, and less notably in CD47 where all the PDSs induced similar expression patterns. The most obvious differences between both breast cancer cell lines were observed in PDL1 and PDL2 genes. Unlike MCF7 cells, MDA-MB-231 cells displayed very high levels of basal PDL1 expression in 2D cultures, and its expression was down-regulated when growing in PDS. And, PDL2 expression was totally absent in MCF7 cells in both, 2D and PDS cultures.

Altogether, these data indicate that PDSs modulate the expression of specific, and selected, immunomarkers in cancer cells growing in these human based cancer microenvironments. Moreover, this modulation varied and depended on each individual PDS. The MCF7 cell line was more sensitive than the MDA-MB-231 cell line to the influence from the various PDS-based microenvironments suggesting that it might be a superior reporter cancer cell line for this type of assay.

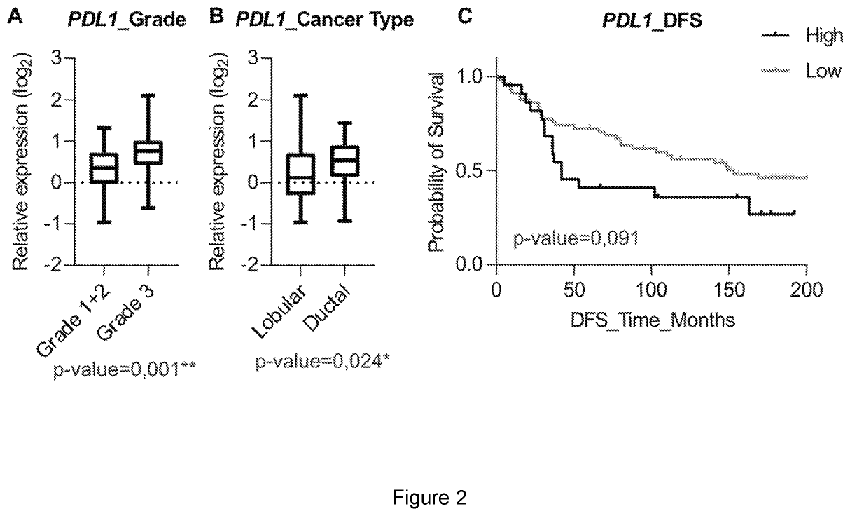

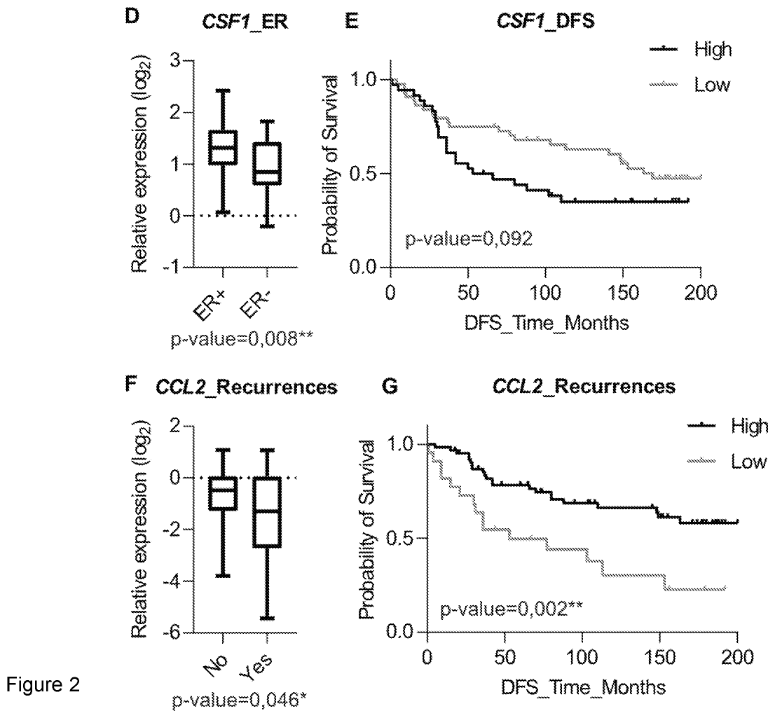

Analysis of the association between immune marker expression in PDSs and clinical variables of the original tumor. We analyzed whether the PDS-modulation of the immune markers correlated to clinical parameters, which were available from the breast cancer donor. The clinical parameters were grade, recurrences, cancer type, estrogen receptor status (ER), progesterone receptor status (PgR), metastasis in lymph nodes as well as patient disease free survival (DFS), expressed as months without recurrences or till death by cancer. Statistical analysis was performed using SPSS to correlate the expression of these immune markers to clinical parameters. Interestingly, changes in three of the analyzed immune regulators, PDL1, CSF1 and CCL2, were associated with clinical characteristics and cancer progression (FIG. 2, Table 2).

PDSs from high-grade tumors (Grade 3) induced higher levels of PDL1 mRNA in MCF7 cells compared to Grade 1 and 2 tumors (FIG. 2A). PDL1 expression was also usually higher in PDSs from ductal carcinoma than lobular (FIG. 2B). Moreover, PDSs from patients with shorter times without relapses or tumor progression showed a trend to induce higher levels of PDL1 in the MCF7 cells (FIG. 2C).

High expression of CSF1 in MCF7 cells growing in PDSs was further significantly associated with ER-positive breast cancer (FIG. 2D). Similar to PDL1, higher levels of CSF1 were induced by PDSs from patients with shorter times without relapses or tumor progression, (FIG. 2E). On the other hand, PDSs inducing high CCL2 expression were generally obtained from patients that had significantly fewer recurrences and longer time without relapses (FIGS. 2F, 2G). Taken together, the expression of PDL1, CSF1 and CCL2 immune regulators in MCF7 cell PDS-cultures mirrored clinical properties of the original breast cancer.

Similar analysis using MDA-MB-231 growing in PDSs did not reveal significant associations between changes in the selected immunomarkers and clinical variables (Table 2). These data support that the ER-positive cancer cell line MCF7 is more sensitive compared to the triple negative cell line MDA-MB-231 regarding monitoring of clinically relevant changes induced by the cancer microenvironment in this model setting.

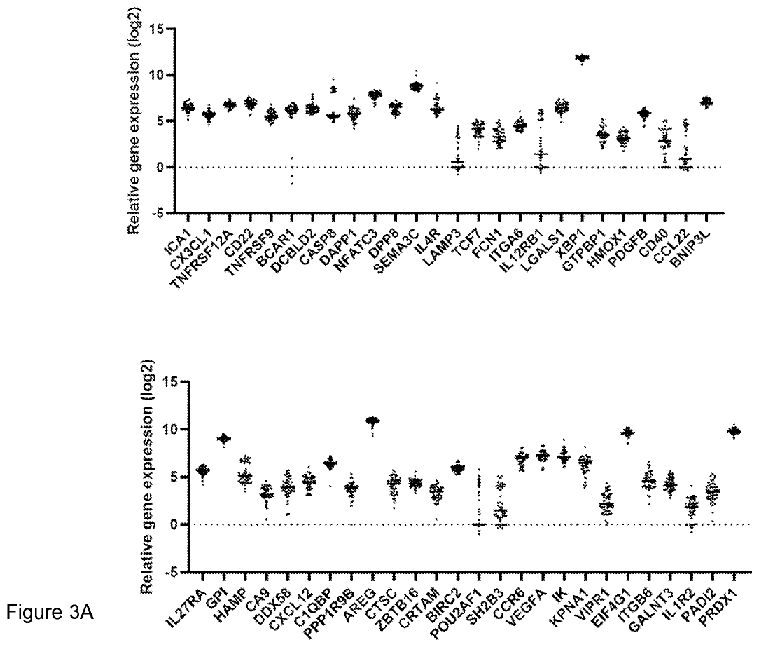

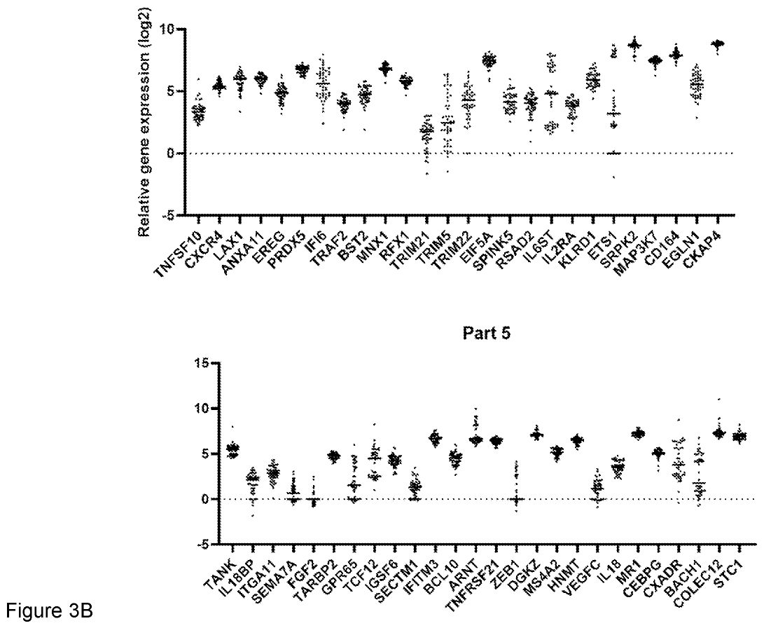

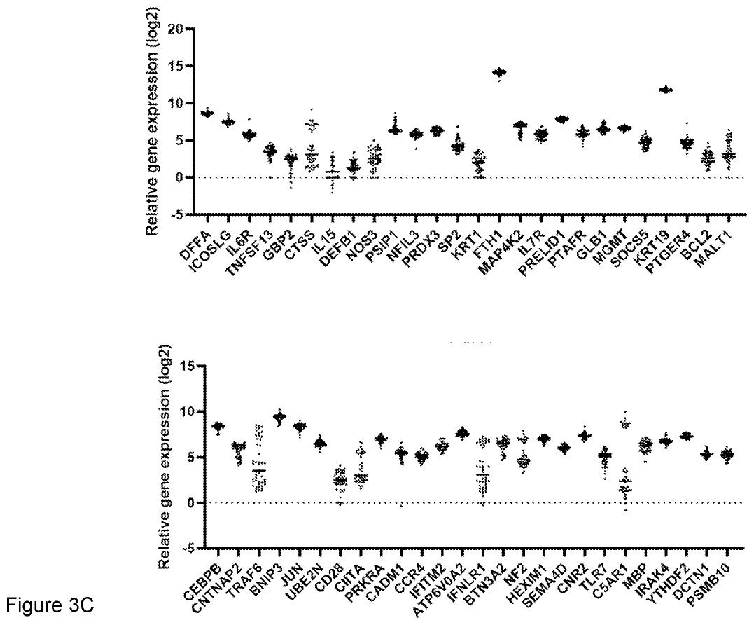

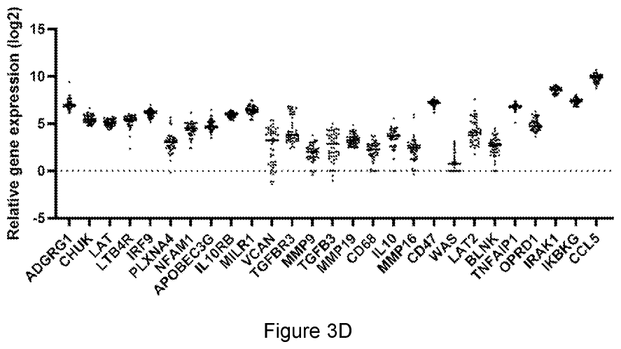

Further analysis of immune markers in breast cancer PDS samples and the association of immune marker expression and clinical variables. The expression of 399 genes related with immune response and modulation were further analyzed by next generation sequencing (NGS) in MCF7 cells that had been growing in a cohort of 48 breast cancer patient-derived scaffolds. 183 genes coding for immunomarkers were found to be expressed in the PDS culture system (FIGS. 3A-3D).

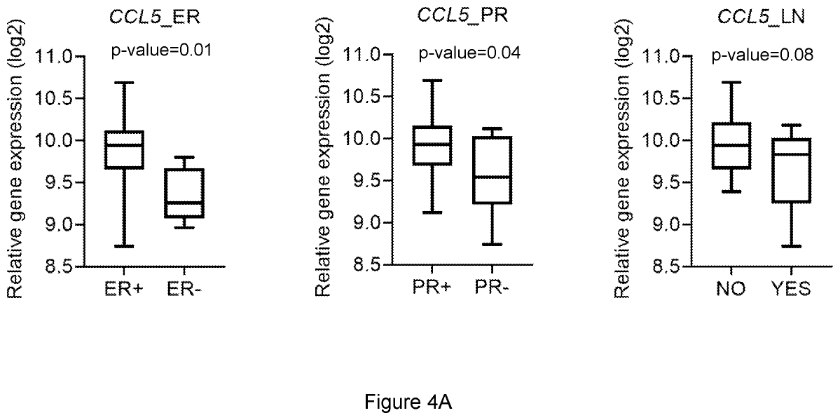

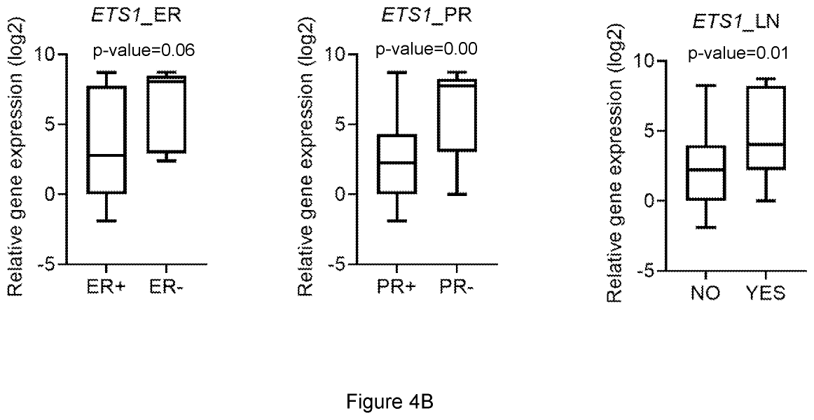

In addition, the differential expression of 44 immune markers found in the PDS samples were significantly associated to clinical variables, such as relapses, grade, estrogen receptor status (ER), progesterone receptor status (PR) and metastasis in lymph nodes (Table 3). Two of these immune markers were CCL5, which is a chemoattractant for blood monocytes, memory T-helper cells and eosinophils, and ETS1, which is a transcription factor directly involved in the expression of cytokine and chemokine genes, associated to clinical variables is plotted in FIGS. 4A and 4B2.

To identify gene expression patterns in the PDS samples, Principal Components Analysis (PCA) was applied to the 183 identified genes from the 48 PDSs (FIGS. 5A-5C). PCA scores in FIG. 5A revealed that the PDSs were distributed in 2 clusters based on the immune marker gene expression. PCA gene loading in FIG. 5B illustrates the contribution of the expression of individual immune markers to the PCA scores in FIG. 5A. Black dots in FIG. 5B indicate gene expression of 30 immune markers which influence the PDSs clustering distribution to a high degree. These 30 genes were then identified using self-organizing map (SOM) and dynamic PCA tools. FIG. 5C shows a heatmap with the gene expression of the 30 immune markers (columns) among the individual PDSs (rows) using average linkage as clustering method and Euclidean distances as the distance measure. The 14 PDSs (columns) grouped on the left coincide with the left cluster in FIG. 5A.

### Example 3—an In Vitro Cancer Model Based on Patient-Derived Tumor Tissue—Colon Cancer

Provided are details of the preparation of an in vitro cancer model based on decellularized patient-derived tumor scaffolds from colon cancer repopulated with colon cancer cells. Provided are also further details of the analysis of the PDS microenvironment and the expression of specific immune-related molecules, which are maintained in the tumor microenvironment even after decellularization.

Patient material. The regional ethical review board in Gothenburg approved this study and informed consent was obtained from all participants (DNR: 590-15). A total of 80 frozen colon cancer tumors were used (primary cancer tissue samples collected after colorectal cancer surgery at the Sahlgrenska University Hospital, Gothenburg, Sweden).

Tumor decellularization and generation of PDS. Colon tumor samples were decellularized as described for breast tumor samples in Example 1.

Protein composition analysis of patient-derived scaffolds by mass spectrometry. Protein composition analysis of the decellularized colon cancer PDS samples by mass spectrometry was performed as described for breast cancer PDS samples in Example 1.

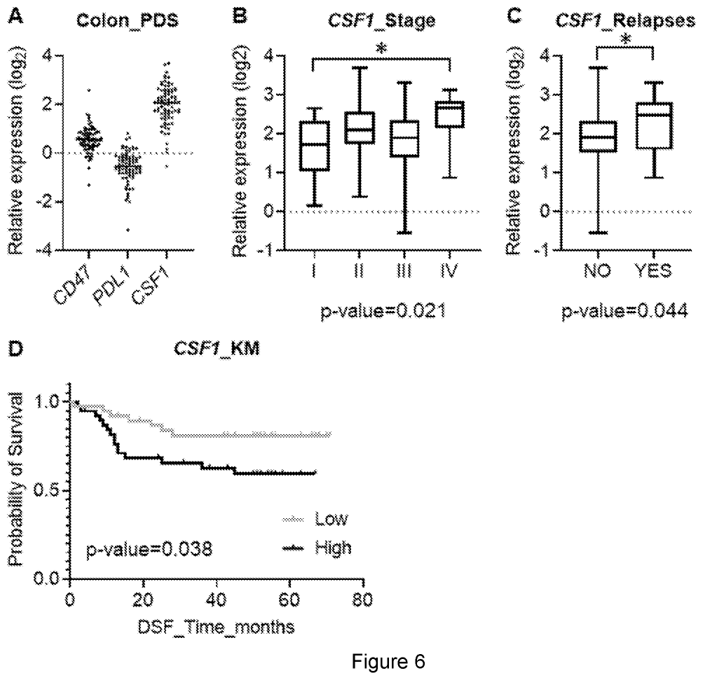

Gene expression analysis of immune markers in cancer cells growing in PDSs. The human colorectal adenocarcinoma cell line HT29 was used to re-cellularize 80 colon cancer PDSs. After 21 days, the gene expression of 4 immuno markers, CD47, PDL1, CSF1 and CCL2, were analyzed by qPCR in the HT29 cells growing in the colon PDSs and compared with T47D cells cultured in 2D (FIGS. 6A-6D). Substantial expression variability of CD47, PDL1 and CSF1 between cells growing in different colon PDSs was observed (FIG. 6A). CCL2 gene expression was not detected. The widest distribution in expression between PDS samples was identified for the CSF1 gene, and the differential expression of this gene was significantly associated to relevant clinical variables and tumor progression. For example, a high expression level of CSF1 detected in HT29 cells growing in colon cancer PDSs was linked to advanced stage, relapses and poor outcome (FIGS. 6B-6D). These results illustrate that PDS samples obtained from other types of cancer, besides breast cancer (Examples 1 and 2), are also able to recapitulate the characteristics and association with clinical variables of the original tumor, and they are, thus, equally suitable for use as in vitro cancer models.

### Example 4—Use of the PDS Model System for Prediction of how Immune Cells Will Interact with Cancer Cells

The PDS model was further used as a growth platform to study its specific effect on the interaction between cancer cells and T lymphocytes (T cells). For this purpose, T cells were added to, and co-cultured with MCF7/Luc cancer cells in PDSs. At specific time points, cells and culture medium was collected and gene expression and viability, as well as secreted IFNγ, were analyzed. The results were then compared to results obtained from co-cultures of T cells and MCF7/Luc cancer cells cultured in 2D (i.e., cells cultured in traditional adherent cultures without the PDS).

Isolation of T lymphocytes (T cells) and activation protocols. Peripheral blood mononuclear cells (PBMCs) were isolated using Ficoll-Paque (GE Healthcare) density gradient separation from healthy donors' buffy coats purchased from the local blood bank T cells were negatively selected using Dynabeads® Untouched™ Human T Cells (Invitrogen, Life Technologies). To test different T cell responses and the T cell killing capacity, a fraction of the isolated T cells was activated. Different grades of T cell activation were achieved using the following protocols:
Mild T cell activation: T cells were incubated for 20 hours with immobilized monoclonal antibody to CD3 (0.1 μg/mL) and soluble anti-CD28 (0.1 μg/mL) in RPMI-1640 media supplemented with 10% heat inactivated FBS. Then, T cells in suspension were recovered by centrifugation 5 min at 500×g and re-suspended in fresh media without antibodies.
Medium T cell activation: Same procedure as for mild activation, but with the addition of IL2 (50 U/1×10⁵ T cells) and without washing away soluble anti-CD28 prior to co-culture with the cancer cells in the PDS.
Strong T cell activation: T cells were incubated for 48 hours with Dynabeads® Human T-Activator CD3/CD28 (Gibco, Life Technologies) following the manufacture's recommendations.

PDSs generated from fresh breast tumors were repopulated with MCF7/Luciferase cancer cells as described in Example 2 and cultured for 21 days. Then, 2×10⁵ non-activated (TNA) or activated T cells (TA-F) were added to the cultures and all cells were co-cultured for an additional 24 (mild and medium activation protocols) or 72 (strong activation protocol) hours. After this time, the medium containing the T cells was recovered and T cells were retrieved by centrifugation. The T cells were then analyzed by FACS and IFNγ was measured in the culture medium. The MCF7/Luciferase cells were detached from the PDSs with Trypsin and viability as well as gene expression changes were analyzed. 2D co-cultures were included as controls by culturing MCF7/Luciferase cells in 24-well plates for 48 hours (until approximately 70% confluency) prior to addition of T cells. T cell co-cultures and analysis were then performed in the same manner for 2D cultures as described above for PDSs.

To characterize the T cells and their activation state, flow cytometry analysis (FACS) was performed on single cells after excluding dead cells with Live/Dead fixable aqua dead cell stain kit (Molecular Probes). Data acquisition was performed on a LSRII flow cytometer (BD Biosciences), equipped with FACS Diva software (BD Biosciences) and analyzed using FlowJo software (TreeStar Inc). The following antibodies were used: CD3-APC/H7 (clone SK7), CD4-AF700 (clone OKT4; Invitrogen), CD8-BUC395 (clone RPA-T8), CD25-APC (clone M-A251), CD127-BV650 (clone HL-7R-M21), PD1-BUV737 (clone EH12.1) and CD69-PE (clone FN50) (BD Biosciences).

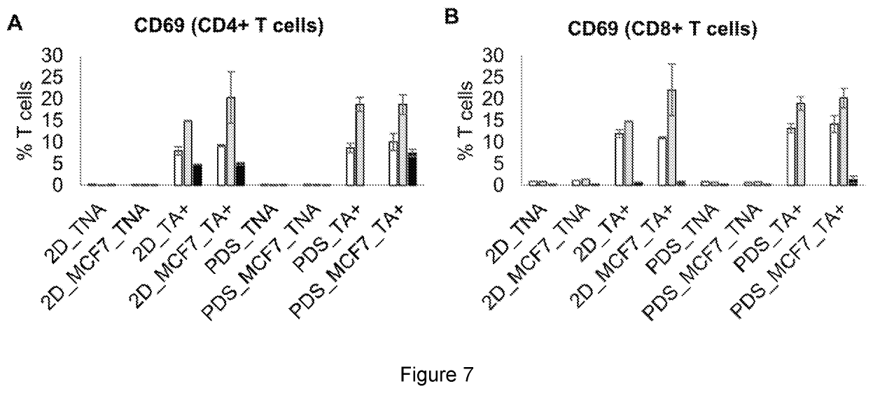

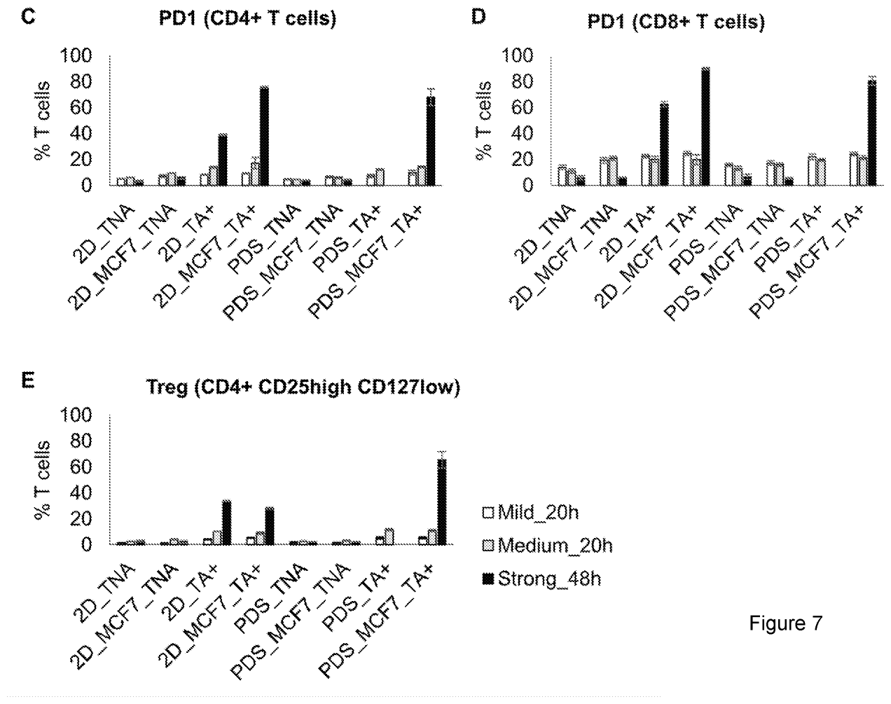

The ability of the cell-free PDSs to activate non-activated T cells was tested (FIG. 7). No significant increase in the expression of the T cell activation marker CD69 was observed in CD4+ or CD8+ T cells following culture with cell-free PDSs for 72 hours (PDS_TNA; FIGS. 7A, 7B) compared with non-activated T cells cultured in regular 2D cultures (2D_TNA). Similar results were observed in the co-culture of non-activated T cells also when MCF7/Luc cells were included in the 2D or PDS culture (2D_MCF7 TNA; PDS_MCF7 TNA), indicating that neither the PDS on its own, nor MCF7/Luc cancer cells presented to T cells in the PDS were able to activate T cells.

In contrast, activation of the T cells by exposure to CD3/CD28 antibodies increased the CD69 marker expression (FIGS. 7A, 7B, 2D_TA+). The activation of T cells was even more pronounced with the medium activation protocol, but, the strong T cell activation protocol instead resulted in a loss of the CD69 marker expression. The loss of CD69 expression after the strong activation protocol could be a consequence of overstimulation, and thereby, exhaustion of T cells, which is also supported by the high % of Treg cells using this activation protocol (FIG. 7E, Strong_48 h). Treg cells are a subpopulation of CD4+, CD25high, CD127low T cells with a role in the feedback control of the magnitude of the immune response and, an increased fraction of Treg cells is associated with sustained and strong activation. When the activated T cells were cultured with the PDS, a slight increase in the percentage of these cells expressing CD69 was consistently observed using the mild activation protocol (PDS_TA+). Also the presence of MCF7/Luc cells affected the expression of CD69 in the pre-activated T cells (MCF7 TA+samples), although the combination of cancer cells and PDS did not have any synergetic effect on the T cells.

PD1 expression also reflects T cell activation, although a high and maintained expression has been associated with a progressive loss of T cells functions. Hence, the T cell PD1 expression was also investigated by FACS. As illustrated in FIGS. 7C, 7D, the percentage of T cells expressing the PD1 receptor was influenced by the presence of cancer cells and pre-activation, but not by the use of PDS. A consistent increase in the percentage of PD1-expressing T cells was observed when non-activated T cells (TNA) had been cultured with MCF7/Luc cells both in 2D and in the PDS (2D_MCF7 TNA, PDS_MCF7 TNA). As expected, the pre-activation of T cells increased the percentage of PD1-positive cells, reaching the highest numbers using the strong activation protocol with 40% and 60% for CD4+ and CD8+ T cells, respectively (2D_TA+). Co-culture of activated T cells with MCF7/Luc cells promoted the increase in the number of PD1-positive cells, especially in strong activation T cells (2 and 1.5-fold change for CD4+ and CD8+ T cells respectively), independently if the T cells were cultured in 2D or PDS (2D_MCF7 TA+, PDS_MCF7 TA+).

Thus, PDSs are able to contribute to the activation of the T cells once they have been pre-activated, as shown by the increase in CD69 expressing T cells; but only the presence of cancer cells influenced the fraction of PD1 expressing T cells.

MCF7/Luciferase cells viability measurements. Cancer cells were detached from PDSs or the plate surface in 2D cultures by 5 min incubation in Trypsin at 37° C. After Trypsin inactivation and removal, cells were re-suspended in 400 μL supplemented RPMI-1640. 300 μL of this solution was used for RNA extraction and gene expression analysis as described in Example 2, and the remaining 100 μL was used for viability measurements by Luciferase luminescence assay. For the Luciferase luminescence assay, serial dilutions of the retrieved cells were plated in 96-well plates (flat bottom clear, white polystyrene), and allowed to settle overnight (37° C., 5% CO₂ humidified atmosphere). 150 μg/mL D-luciferin substrate was then added immediately prior the light emission measurement in a GloMax® Discover Microplate Reader (Promega). In each plate, a standard curve was generated using MCF7/Luciferase cells from fresh 2D cultures by calculating the number of cells by light emission units.

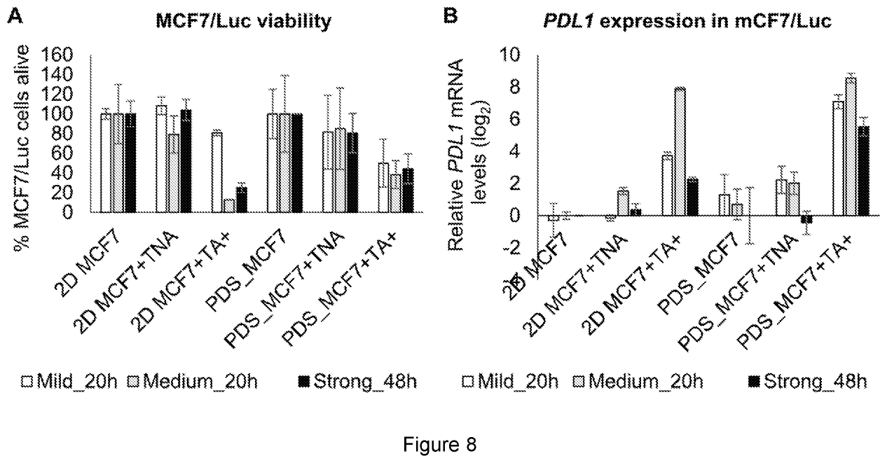

After having been co-cultured with T cells in PDS, MCF7/Luc cells were harvested and the amount of viable cells was quantified by measuring the emission of luminescence in a luciferase assay (FIG. 8A). MCF7/Luc cells cultured in PDS or in 2D without the addition of T cells were used as a control (100% viable cells). In most of the samples analyzed, no changes in viability of the MCF7/Luc cells were observed in 2D co-cultures with non-activated T cells (2D_MCF7 TNA); but the viability dropped in an activation grade dependent manner using activated T cells (2D_MCF7 TA-F). Mild activation showed only a reduction of 20% in viability while there was a 75-85% reduction in viable cells using the two strong activation protocols. The co-cultures in PDS with non-activated T cells (PDS_MCF7 TNA) showed a 20% consistent but not statistically significant reduction in MCF7/Luc viability. Nevertheless, in PDS co-cultures using activated T cells (PDS_MCF7 TA-F), only 25%-50% of the MCF7/Luc cells remained viable. Surprisingly, the differences in killing capacity between mild and strong activations of the T cells were less obvious using PDS co-cultures compared to 2D cultures. Co-cultures with PDS further showed higher variability in general compared to 2D as expected, because of the heterogeneity of the different in vivo based patient tumor microenvironments.

Changes in gene expression in MCF7/Luc cells growing in PDS and co-cultured with T cells. Gene expression analysis was performed as mentioned in Example 2. Firstly, the expression of the immunomarker PDL1, which is involved in the T cells immunomodulation by the cancer cells, was analyzed (FIG. 8B). Although there was a slight increase in PDL1 expression levels of MCF7/Luc cells after co-cultures with non-activated T cells (TNA), the most pronounced upregulation was observed in co-cultures using activated T cells (TA+), and the increase in PDL1 expression was further significantly higher in most of the cases using PDS as co-culture platform. These results indicate that the exposure of MCF7/Luc to T cells increases cancer cell PDL1 expression through gene activation, or by enrichment of PDL1 positive cancer cells which are not susceptible to T cell killing. However, only contact with pre-activated T cells triggered a clear PDL1 upregulation in the MCF7/Luc cancer cells.

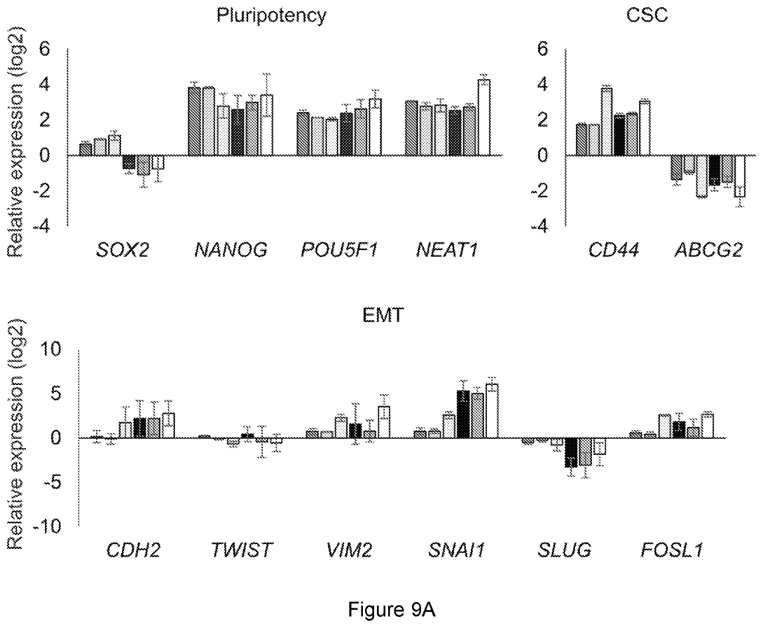

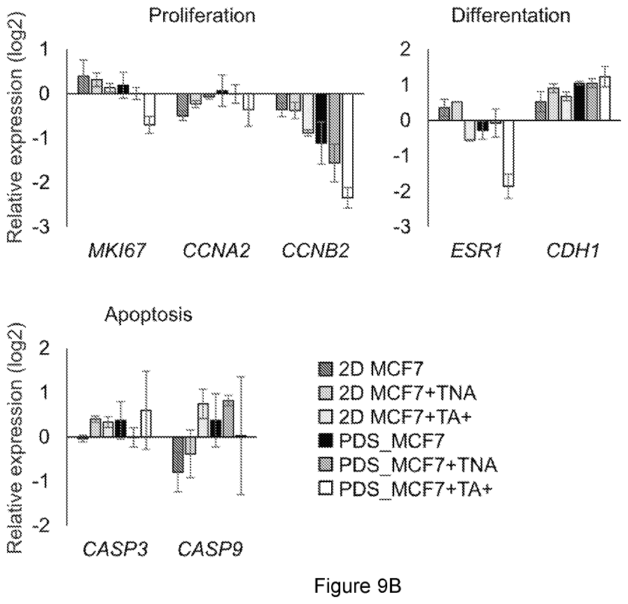

Moreover, to determine if the exposure to T cells by MCF7/Luc cancer cells growing in PDS would influence the gene expression profile of the surviving MCF7/Luc cells, we analyzed the expression of a gene panel representing breast cancer-related processes such as proliferation (MKI67, CCNA2, CCNB2), differentiation (ESR1, CDH1), pluripotency (SOX2, NANOG, POU5F1, NEAT1), EMT (CDH2, TWIST, VIM2, SNAI1, SLUG, FOSL1), cancer stem cell markers (CSC) (CD44, ALDH1A3, ABCG2) and apoptosis (CASP3, CASP9) in the harvested MCF7/Luc cells (FIGS. 9A and 9B) (Akrap et al., 2016). Co-culture of MCF7/Luc cells with non-activated T cells for 72 hours did not significantly affect the expression of any of the analyzed genes, neither in 2D nor in PDS cultures. Changes in expression levels of some of the genes were however detected when strongly activated T cells (TA+) (48 hours with CD3/CD28 Dynabeads) were used for the co-cultures, coinciding with the partial killing of the MCF7/Luc population by the T cells. The remaining viable MCF7/Luc population displayed a lower expression of the proliferation markers MKI67 and CCNB2, and the differentiation marker ESR1; together with an increased expression of the pluripotency genes POU5F1 and NEAT1, the CSC-related gene CD44, and the EMT markers VIM2, SNAI1 and FOSL1. Similar profiles were obtained for both 2D and PDS co-cultures, although changes were in general more pronounced in the PDS cultures. Thus, these results suggest that after 72 hours of co-culture, strongly activated T cells mainly killed the proliferative cancer cell population, and the surviving MCF7/Luc population was enriched for cells with pluripotency and cancer stem cell features.

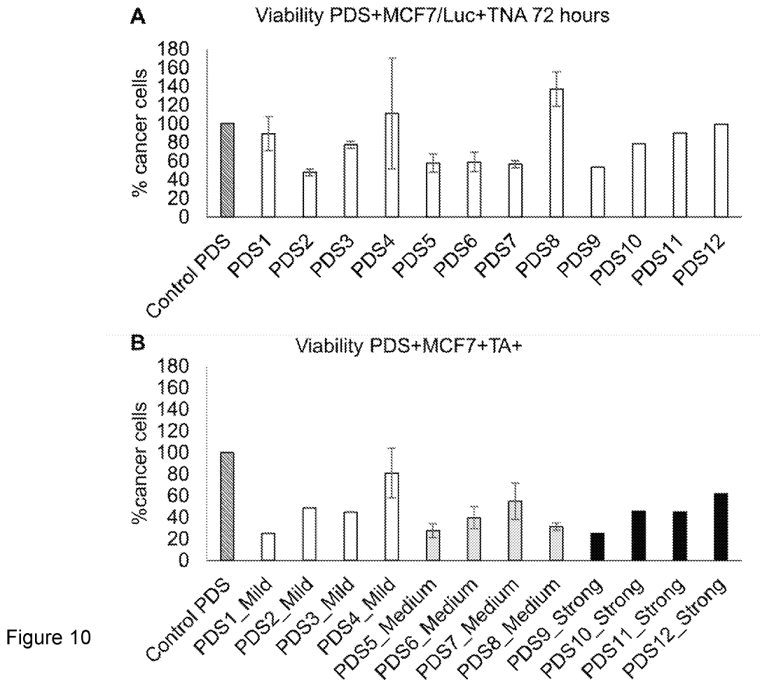

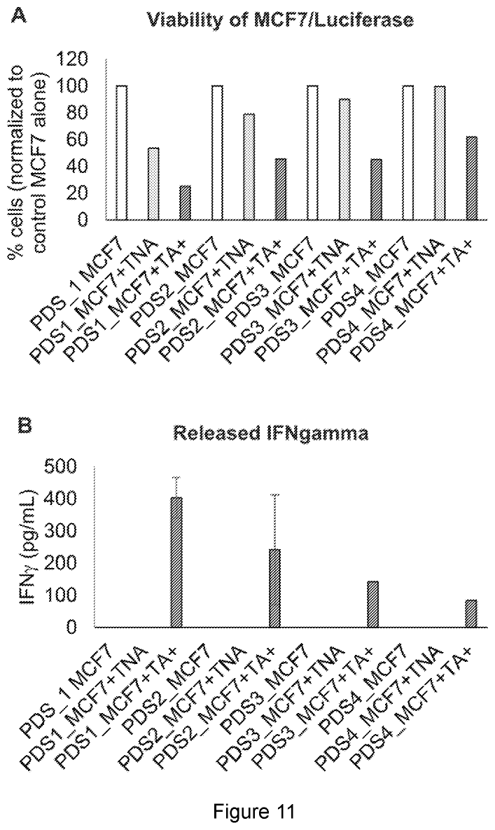

Analysis of the influence of the patient-specific PDS tumor microenvironment in the co-cultures with cancer cells and T cells. Due to the pronounced heterogeneity within the cancer microenvironment and the variety between patients, we next analyzed the effect of each of four individual PDSs using co-cultures with MCF7/Luc cells and T cells (FIG. 10). Each PDS represents a combination of activities of several different cell types within the cancer microenvironment, and therefore the individual PDS is able to mediate specific actions, including various immune responses. This was also obvious from the results of our previous analysis of immunomarker expression in PDSs from biobanked-tumors (as described in Example 2). Interestingly, MCF7/Luc cell viability measurements in each PDS revealed substantial differences in sensitivity to the non-activated T cells, where only around 50% of MCF7/Luc cells growing in PDS2 and PDS9 had survived after 72 hours in co-cultures with non-activate T cells (TNA), while there was almost no effect on viability in several of the PDSs (PDS1, PDS4, and PDS12; 100% viability) or even an increased growth in one PDS (PDS8) (FIG. 10A). There was also a distinct difference in sensitivity to activated T cells (TA+) in the various PDSs, spanning from 25% of live cells in PDS9 to 81% in PDS4 (FIG. 10B). Unexpectedly, the difference in killing by the activated T cells (TA+) was not associated with intensity of the pre-activation protocol (i.e., mild to strong) or the co-culture incubation time. These data, thus, indicated that a certain grade of activation of the T cells is enough for an increase in the killing capacity of the T cells in the PDS co-cultures, and that most of this killing activity occurs during the first 24 hours of co-culture. Additionally, when measuring the amount of IFNγ that was released into the medium by strongly activated T cells and 4 PDSs (FIG. 11), the data supported the differences in killing and viability by the activated T cells (TA+) observed in the different PDSs. Despite a substantial killing of cancer cells by the T cells in some PDS co-cultures using non-activated T cells (TNA), only activated T cells (TA+) were able to induce detectable IFNγ in the medium, suggesting that different T cell killing mechanisms are acting for the non-activated versus activated T cells, respectively.

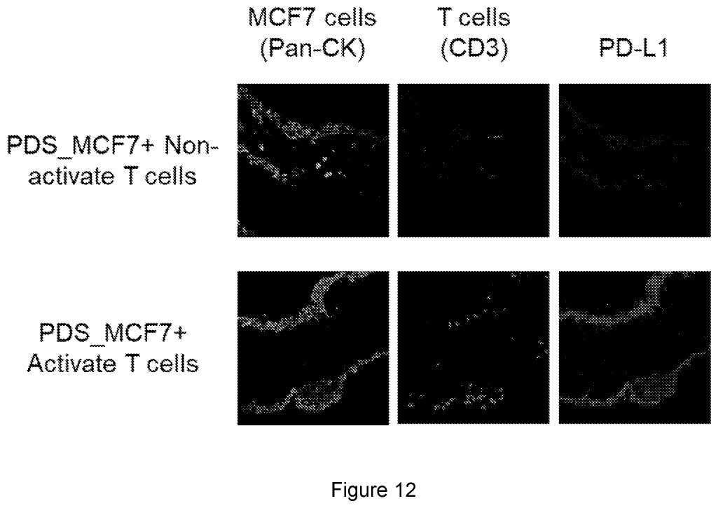

FIG. 12 supports this data illustrating, using immunofluorescence microscopy, how active T-lymphocytes were able to infiltrate the cancer cells growing in breast cancer PDSs. FIG. 12 also shows the increase in PDL1 protein expressed by the cancer cells only when in presence of active T-lymphocytes.

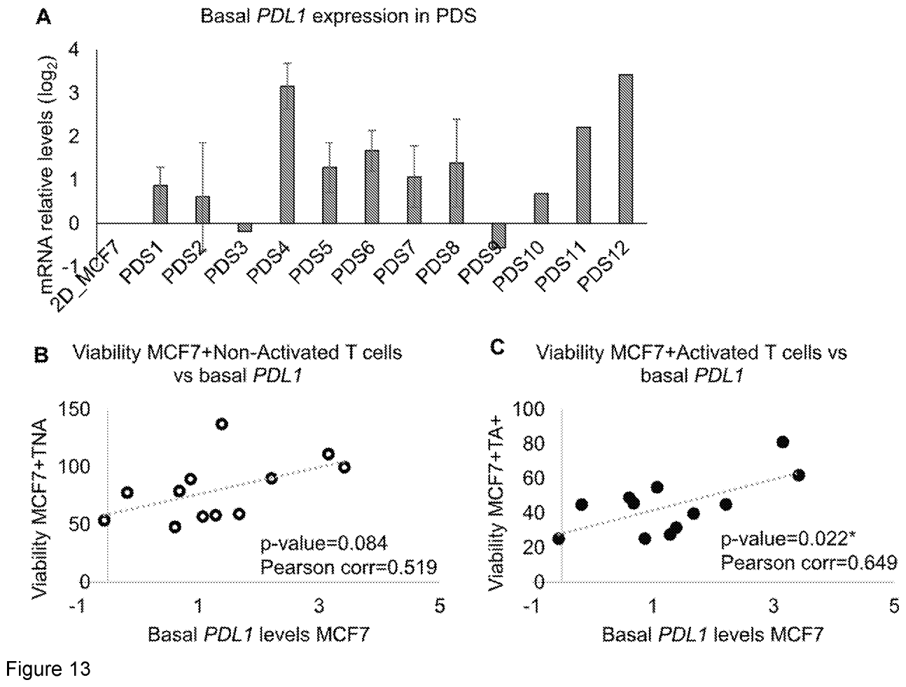

When analyzing the basal expression level of PDL1 mRNA in MCF7/Luc cells growing in the different PDSs without being in contact with T cells, we observed large variation in PDL1 levels (FIG. 13A). Interestingly, Spearman's correlation analysis indicated that these basal PDL1 levels induced by the cancer microenvironment in the PDS-cultures was positively correlated with the survival capacity after 72 hours in co-culture with non-activated T cells (Table 4) (p-value=0.017). MCF7/Luc growing in PDS with higher basal PDL1 expression were showing a trend of higher viability in co-cultures with non-activated (TNA) (FIG. 13B). Spearman's correlation was not significant in co-cultures with activated T cells (TA+) but showed a similar trend as for the non-activated T cells (FIG. 13C). Hence, the PDL1 expression triggered by each PDS might modulate the susceptibility of the cancer cells to be killed by the T cells.

TABLE 4
Spearman's correlations between PDL1 expression in MCF7/Luc cancer cells and
their susceptibility to be killed by T cells in PDS co-cultures. (*p-value < 0.05).

| | | | PDL1 | | | | |
|---|---|---|---|---|---|---|---|
| | | | Expression | | | | |
| | | | Basal | | | Viability | |
| | | | PDL1 | PDL1 | PDL1 | Viability | Viability |
| | | | MCF7 | MCF7_TNA | MCF7_TA+ | MCF7_TNA | MCF7_TA+ |
| PDL1 | Basal PDL1 | Correlation | 1 | | | | |
| Expression | MCF7 | Coefficient | | | | | |
| | | p-value | . | | | | |
| | | N | 12 | | | | |
| | PDL1 | Correlation | 0.5 | 1 | | | |
| | MCF7_TNA | Coefficient | | | | | |
| | | p-value | 0.117 | . | | | |
| | | N | 11 | 11 | | | |
| | PDL1 | Correlation | 0.238 | 0.4 | 1 | | |
| | MCF7_TA+ | Coefficient | | | | | |
| | | p-value | 0.457 | 0.223 | . | | |
| | | N | 12 | 11 | 12 | | |
| Viability | Viability | Correlation | 0.671* | 0.318 | −0.098 | 1 | |
| | MCF7_TNA | Coefficient | | | | | |
| | | p-value | 0.017 | 0.34 | 0.762 | . | |
| | | N | 12 | 11 | 12 | 12 | |
| | Viability | Correlation | 0.428 | 0.26 | −0.193 | 0.193 | 1 |
| | MCF7_TA+ | Coefficient | | | | | |
| | | p-value | 0.165 | 0.44 | 0.548 | 0.548 | . |
| | | N | 12 | 11 | 12 | 12 | 12 |

### Example 5—Use of the PDS Model System for Analysis and Identification of Macrophage Polarization

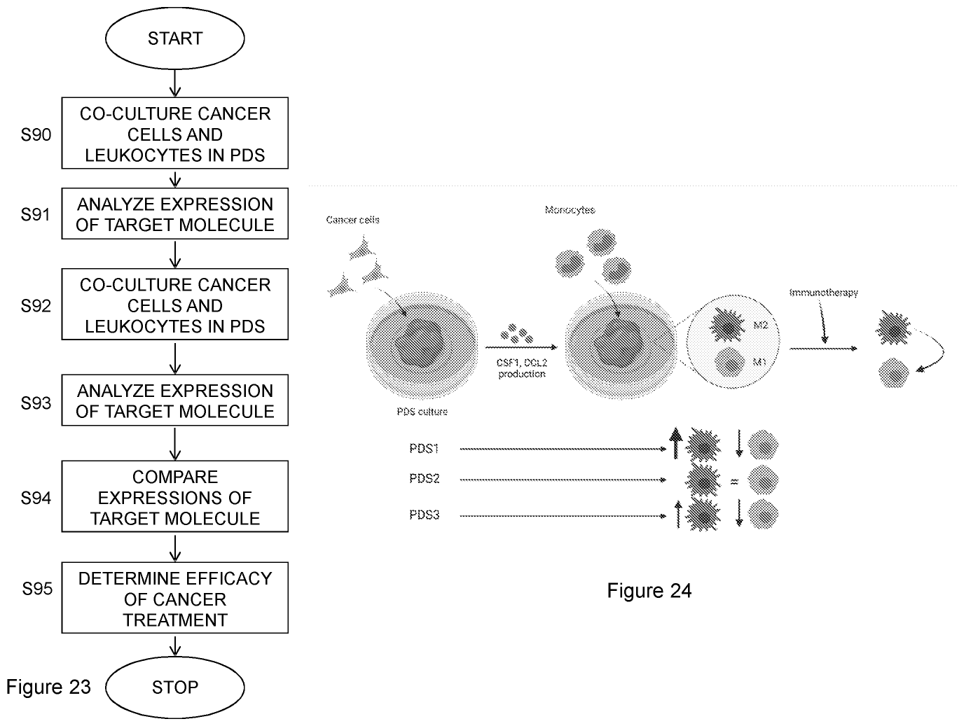

The data obtained using patient-derived scaffolds strongly indicate that PDSs have the capacity to affect also other immune cells than T-lymphocytes. For instance, we hypothesize that the PDS samples will be able to induce macrophage polarization in a patient-dependent manner, which will enable the analysis of macrophage influence on both cancer cells and/or T lymphocytes. Such data will further enable screening of treatments that target the identified tumor promoting interactions (FIG. 24). The basis for this hypothesis lies in the findings, as presented in FIGS. 1 and 2 for breast cancer PDSs, and in FIG. 6 for colon cancer PDSs, where a differential expression of two cytokines genes, CSF1 and CCL2, is shown. These genes are essential for the recruitment and survival of monocytes, and a differential expression of these genes was identified, and was associated with relevant clinical variables. Differential expression of these genes, which is dependent on the specific PDS samples, will, thus, determine the polarization of macrophages, and their subsequent interaction with the cancer cells as grown in the PDS. The PDS system can, thus, be used as testing platform for therapies targeting also macrophage polarization. Also, it will provide predictive information for specific patients by including therapy screens, and contribute to the design of new personalized treatment strategies by targeting the tumor-associated macrophage (TAM) mediated immune suppression.

### Example 6—Use of the PDS Model System for Prediction of Immune Therapy Efficacy

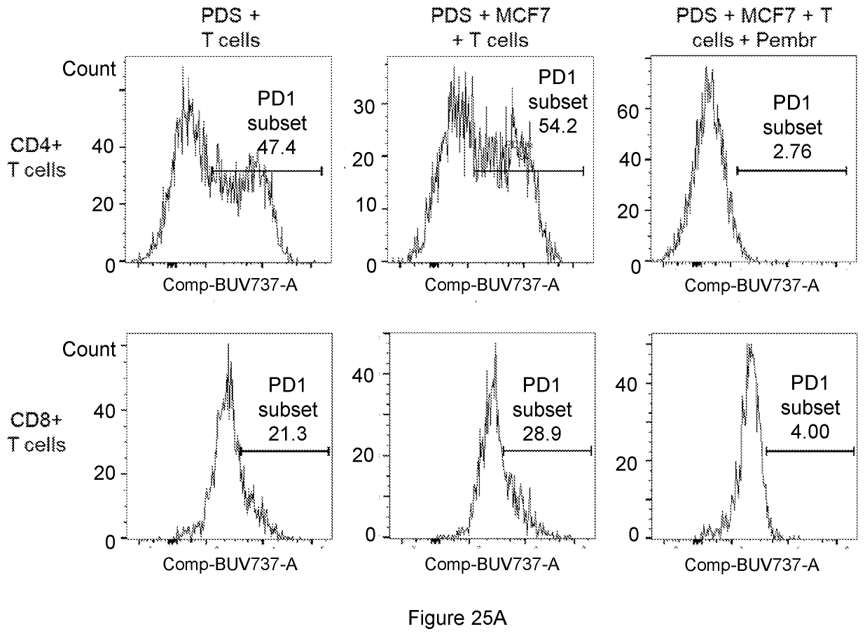

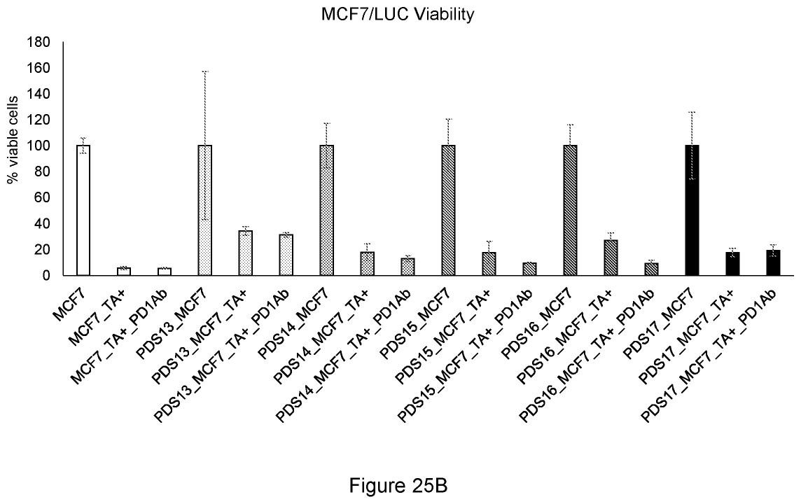

In previous Examples, it was demonstrated that breast cancer PDS samples are suitable for cancer cells and T-lymphocyte co-cultures. It was found that when T-lymphocytes were activated and gene expression of the PDL1 gene was induced by the MCF7 cancer cells, T-lymphocytes were able to kill the cancer cells. In addition, in FIG. 13, it is shown how differential expression of PDL1 by MCF7 cells is triggered by different breast cancer PDSs. This figure also shows that different PDSs induced differential susceptibility of cancer cells to be killed by T-lymphocytes, as well as correlations between PDL1 expression and killing susceptibility. This data shows the suitability of the PDS system for testing immune therapies such as PDL1-PD1 blockade treatments. Based on this, we analyzed the effect of a well-known immune therapy, a PD1-blocking antibody, Pembrolizumab, in the PDS model system (FIG. 25). First, T-lymphocytes were activated 20 hours by exposure to CD3/CD28 Dynabeads. Then, and prior to being added to the PDS cultures, some of the T-lymphocytes were incubated with Pembrolizumab (Pembr or PD1Ab) for 30 min (100 μg/mL in 1×10⁶ T-lymphocytes/mL suspension). The blockade of PD1 protein in the T-lymphocytes was confirmed by FACS (FIG. 25A). The T-lymphocytes, with or without PD1 blockade (Pembr) were then added to the PDS cultures, in which the MCF7 cells had been growing for 21 days. The co-cultures were incubated for 48 hours. Control samples included samples with MCF7 cells or T-lymphocytes on their own, and 2D cultures. Once being added to the cancer cells growing in the PDSs, the PD1 blockade of the T-lymphocytes was investigated by analyzing and comparing the viability of the MCF7 cells in the different samples, and PD1 blockade by Pembrolizumab was found to cause differential and PDS-dependent effects. In some PDSs, such as in PDS14, PDS15, and PDS16, the killing effect of the T-lymphocytes was higher after incubation with Pembrolizumab, whereas in other PDSs no changes in the killing efficacy was observed by the T-lymphocytes after Pembrolizumab treatment as compared to without this treatment (such as in PDS13 and PDS17) (FIG. 25B, Table 5).

TABLE 5
Table showing the % of viable MCF7 cells after 48 hours
of co-culture with T-lymphocytes in 5 different PDSs.

| | Control | MCF7 + | MCF7 + |
|---|---|---|---|
| | (only MCF7) | TA+ | TA + Pembr |
| 2D MCF7 | 100.0 ± 5.7 | 5.7 ± 1.2 | 5.5 ± 0.6 |
| PDS13 | 100.0 ± 57.0 | 34.2 ± 3.4 | 31.2 ± 1.7 |
| PDS14 | 100.0 ± 17.1 | 17.8 ± 6.4 | 12.8 ± 2.3 |
| PDS15 | 100.0 ± 20.2 | 17.6 ± 8.5 | 9.5 ± 0.6 |
| PDS16 | 100.0 ± 16.1 | 27.0 ± 5.8 | 9.1 ± 2.5 |
| PDS17 | 100.0 ± 25.8 | 17.5 ± 3.1 | 19.2 ± 4.3 |

This data shows that the PDS model system is suitable for the analysis and identification of immune treatment efficacy and that the PDS model system can be used to indicate if a specific immune treatment would be suitable for a patient, i.e., the patient from which the tumor was obtained to prepare the PDS sample.

The embodiments described above are to be understood as a few illustrative examples of the present invention. It will be understood by those skilled in the art that various modifications, combinations and changes may be made to the embodiments without departing from the scope of the present invention. In particular, different part solutions in the different embodiments can be combined in other configurations, where technically possible.

### REFERENCES

Akrap N, et al., Identification of Distinct Breast Cancer Stem Cell Populations Based on Single-Cell Analyses of Functionally Enriched Stem and Progenitor Pools. Stem Cell Reports. 6(1): 121-136 (2016).
Landberg G, et al., Patient-derived scaffolds uncover breast cancer promoting properties of the microenvironment. Biomaterials. 235: 119705 (2020).
Sobral-Leite M, et al., Assessment of PD-L1 expression across breast cancer molecular subtypes, in relation to mutation rate, BRCA1-like status, tumor-infiltrating immune cells and survival. Oncoimmunology. 7(12): e1509820 (2018).

1. A method of providing an in vitro cancer model, the method comprising:
selecting (S1) a cell-free scaffold obtained from a tumor in a subject, the tumor having at least one defined tumor property and the cell-free scaffold is selected based on the at least one defined tumor property;
selecting (S2) cancer cells based on the selected cell-free scaffold, wherein the selected cancer cells are not obtained from the tumor, from which the cell-free scaffold is obtained; and
culturing (S3) the selected cancer cells in the selected cell-free scaffold.

2. The method according to claim 1, further comprising analyzing the expression of at least one immunomarker in the cancer cells cultured in the selected cell-free scaffold.

3. The method according to claim 1 or 2, further comprising:
exposing the cancer cells to a cancer treatment while culturing the cancer cells in the selected cell-free scaffold; and
determining a response of the cancer cells to the cancer treatment, preferably a cancer immunotherapy.

4. The method according to claim 3, further comprising.
analyzing the expression of at least one immunomarker in the cancer cells cultured in the selected cell-free scaffold prior to exposing the cancer cells to the cancer treatment;
analyzing the expression of the at least one immunomarker in the cancer cells cultured in the selected cell-free scaffold after exposing the cancer cells to the cancer treatment; and
comparing the expression of the at least one immunomarker in the cancer cells cultured in the selected cell-free scaffold prior to exposing the cancer cells to the cancer treatment with the expression of the at least immunomarker in the cancer cells cultured in the selected cell-free scaffold after exposing the cancer cells to the cancer treatment, wherein determining the response of the cancer cells comprises determining the response of the cancer cells to the cancer treatment based on the comparison.

5. The method according to claim 3, further comprising:
determining the viability of the cancer cells cultured in the selected cell-free scaffold prior to exposing the cancer cells to the cancer treatment;
determining the viability of the cancer cells cultured in the selected cell-free scaffold after exposing the cancer cells to the cancer treatment; and
comparing the viability of the cancer cells cultured in the selected cell-free scaffold prior to exposing the cancer cells to the cancer treatment with the viability of the cancer cells cultured in the selected cell-free scaffold after exposing the cancer cells to the cancer treatment, wherein determining the response of the cancer cells comprises determining the response of the cancer cells to the cancer treatment based on the comparison.

6. The method according to claim 3, further comprising:
determining at least one cancer cell property of viable cancer cells after exposing the cancer cells to the cancer treatment, wherein
the cancer cell property is preferably selected from the group consisting of cancer stem cell-ness, proliferation, migration, apoptosis, epithelial-to-mesenchymal transition, therapy resistance, cell metabolism, cell-to-cell communication, and differentiation; and
determining the response of the cancer cells comprises determining the response of the cancer cells to the cancer treatment based on the at least one determined cancer cell property.

7. The method according to claim 6, wherein determining at least one cancer cell property comprises:
analyzing the expression of at least one cancer cell property marker in viable cancer cells after exposing the cancer cells to the cancer treatment; and
determining the at least one cancer cell property based on the expression of the least one cancer cell property marker.

8. The method according to claim 1, further comprising determining the at least one tumor property of the tumor, from which the cell-free scaffold is obtained.

9. The method according to claim 8, wherein determining the at least one tumor property comprises determining at least one tumor property selected from the group consisting of tumor malignancy grade, cancer type, tumor location, differentiation capability, proliferation capability, infiltration capability, metastasizing capability, epithelial-mesenchymal transition (EMT) capability and cancer stem cell (CSC) capability of the tumor, from which the cell-free scaffold is obtained,
selecting (S1) a cell-free scaffold obtained from a tumor in a subject, the tumor having at least one defined tumor property and the cell-free scaffold is selected based on the at least one defined tumor property;

10. The method according to claim 1, wherein
selecting (S2) the cancer cells comprises selecting a cancer cell line or a primary cancer cell population based on the selected cell-free scaffold; and
culturing (S3) the selected cancer cells comprises culturing cancer cells of the selected cancer cell line or the primary cancer cell population in the selected cell-free scaffold.

11. The method according to claim 1, wherein selecting (S2) the cancer cells comprises selecting cancer cells being sensitive to the influence of microenvironment of the selected cell-free scaffold.

12. The method according to claim 11, wherein selecting (S2) the cancer cell comprises selecting cancer cells having an inducible expression of at least one immunomarker, wherein expression of the at least one immunomarker is altered by the microenvironment of the selected cell-free scaffold.

13. A method of identifying an immunotarget for cancer therapy for a subject, the method comprising:
culturing (S10) cancer cells in a cell-free scaffold obtained from a tumor in the subject;
analyzing (S11) expressions of immunomarkers in the cancer cells; and
selecting (S12) at least one immunomarker as an immunotarget for cancer therapy for the subject based on the expressions of immunomarkers in the cancer cells.

14-21. (canceled)

22. A method of determining at least one tumor property of a tumor in a subject, the method comprising:
culturing (S30) cancer cells, preferably breast cancer cells, in a cell-free scaffold obtained from a tumor in the subject;
analyzing (S31) the expression of at least one immunomarker selected from the group consisting of programmed death ligand 1 (PDL1), colony stimulating factor 1 (CSF1) and chemokine C—C motif ligand 2 (CCL2) in the cancer cells; and
determining (S32) the at least one tumor property based on the expression of the at least one immunomarker.

23-45. (canceled)

46. The method according to claim 1, further determining susceptibility of a tumor to immunotherapy, the method comprising:
selecting (S1) a cell-free scaffold obtained from a tumor in a subject, the tumor having at least one defined tumor property and the cell-free scaffold is selected based on the at least one defined tumor property;
co-culturing (S40) cancer cells and leukocytes in the cell-free scaffold obtained from a tumor in a subject;
determining (S42) the viability of the cancer cells; and
determining (S43) the susceptibility of the tumor in the subject to immunotherapy based on the determined viability of the cancer cells.

47. The method according to claim 46, further determining at least one tumor property of a tumor in a subject, the method comprising:
co-culturing (S50) cancer cells and leukocytes in the selected cell-free scaffold obtained from a tumor in a subject;
analyzing (S51) the expression of at least one tumor property marker in the viable cancer cells; and
determining (S52) at least one tumor property based on the expression of the at least one tumor property marker in the viable cancer cells co-cultured with the leukocytes in the cell-free scaffold,
wherein determining the susceptibility of the tumor in the subject to cancer treatment is based on the determined at least one tumor property.

48. The method according to claim 47, wherein the tumor has at least one defined tumor property selected from the group consisting of tumor malignancy grade, cancer type, tumor location, differentiation capability, proliferation capability, infiltration capability, metastasizing capability, epithelial-mesenchymal transition (EMT) capability and cancer stem cell (CSC) capability.

49. The method according to claim 1, further determining susceptibility of a tumor to immunotherapy, the method comprising:
selecting (S1) a cell-free scaffold obtained from a tumor in a subject, the tumor having at least one defined tumor property and the cell-free scaffold is selected based on the at least one co-culturing (S60) cancer cells and leukocytes in the cell-free scaffold obtained from a tumor in a subject;
analyzing (S62) the expression of at least one immunomarker in the cancer cells from the co-culture of cancer cells and leukocytes in the cell-free scaffold; and
determining (S63) the susceptibility of the tumor in the subject to immunotherapy based on the expression of the at least one immunomarker.

50. The method according to claim 49, further comprising:
culturing cancer cells in a cell-free scaffold obtained from a tumor in the subject;
analyzing the expression of the at least one immunomarker in the cancer cells from the culture of cancer cells in the cell-free scaffold; and
comparing the expression of the at least one immunomarker in the cancer cells from the culture with the expression of the at least one immunomarker in the cancer cells from the co-culture, wherein determining (S63) the susceptibility comprises determining the susceptibility of the tumor in the subject to immunotherapy based on the comparison.

51. The method according to claim 49, further comprising determining at least one tumor property of viable cancer cells co-cultured with the leukocytes in the cell-free scaffold, wherein determining (S63) the susceptibility comprises determining the susceptibility of the tumor in the subject to immunotherapy based on the expression of the at least one immunomarker and based on the at least one tumor property.
